(19) United States
(12) Patent Application Publication
(10) Pub. No.: US 2008/0033546 A1
(43) Pub. Date: Feb. 7, 2008

(54) Methods and apparatus for improving vision

(76) Inventor: Junzhong Liang, Fremont, CA (US)

Correspondence Address: LAW OFFICE OF HARRY J. MACEY, COURT HOUSE PLAZA, SUITE 410, 260 SHERIDAN AVENUE, PALO ALTO, CA, 94306, US

(21) Appl. No.: 11/809,021

(22) Filed: May 30, 2007

Related U.S. Application Data
(60) Provisional application No. 60/810,035, filed on May 31, 2006.
(60) Provisional application No. 60/834,242, filed on Jul. 28, 2006.
(60) Provisional application No. 60/850,927, filed on Oct. 10, 2006.
(60) Provisional application No. 60/854,008, filed on Oct. 23, 2006.

(51) Int. Cl. A61F 2/16; A61F 2/14

(52) U.S. Cl. 623/5.11; 623/6.11

(57) ABSTRACT

Transmittance of light through a pupil or to the retina of a patient's eye is controlled to improve night vision. In one example, this involves providing an ocular device in the form of a contact lens having a central, disk shaped clear window having a diameter that is custom designed based high-order aberrations in an eye and less than the diameter of the patient's pupil at night. The ocular device also has an annular portion that surrounds the disk shaped window portion. The annular portion comprises material that provides reduced light transmittance to provide controlled light transmission at pupil periphery at night and to the retina of a patient. Such controlled light transmittance can reduce photon noise that otherwise can exacerbate halos and ghosts that one can experience at night and/or it can improve night vision contrast. In the case of a contact lens and corneal inlay, light transmittance is controlled across the cornea. In the case of an intraocular lens, light transmittance is controlled across the pupil.

### CROSS-REFERENCE

This application claims the benefit of each of U.S. Provisional Application Nos. 60/810,035, filed May 31, 2006 and entitled Methods and Systems for Refractive Corrections with Enhanced Night Vision, 60/834,242, filed Jul. 28, 2006 and entitled Refractive Vision Corrections with Wavefront Optimized Lenses, 60/850,927, filed Oct. 10, 2007 and entitled Lens of Improved Night Vision and Method of Making Same, 60/854,008, filed Oct. 23, 2006 and entitled Improved Implantable Ophthalmic Lenses, all of which applications are incorporated herein by reference.

### FIELD OF THE INVENTION

The invention generally relates to methods and apparatus for improving vision, including night vision and/or treating myopia, hyperopia or prebyopia.

### BACKGROUND OF THE INVENTION

Even though it is possible to correct high-order aberrations in human eyes with adaptive optics in the laboratories, effective correction of eye's high-order aberrations is still a challenging task for clinical procedures such as wavefront-guided laser vision corrections, wavefront-guided contact lenses, and wavefront-guided spectacles. Clinical procedures can hardly match the performances of an adaptive optics system in precision (less than one tenth of wavelength), in wavefront registration between the wavefront measurement and the wavefront correction, and in closed-loop control of uncorrected wavefront errors. Wound-healing is another factor limiting the success of wavefront-guided laser vision corrections.

In general, glasses, contact lenses, and intraocular lenses are devices used to correct low order aberrations in a person's eye(s) and only correct sphero-cylinder errors. High order aberrations are the aberrations that are not low order aberrations. Without an effective means for correcting high-order aberrations in the eye, many eyes suffer from aberration-induced symptoms such as glare, halo, ghost images, and starburst. The issue of night symptoms is particularly significant for eyes after refractive surgeries. High order aberrations can exist before or arise after refractive surgery (e.g., laser refractive surgery such as PRK or LASIK).

FIGS. 1A-D illustrate two examples of eyes that received refractive surgeries. FIGS. 1A and 1B depict one post-op PRK (Photorefractive Keratectomy) eye and FIGS. 1C and 1D depict one post-op RK (Radial Keratotomy) eye. FIGS. 1B and D show images of wavefront measurements using a Hartmann-Shack wavefront sensor for the two surgical eyes of FIG. 1A FIG. 1B, where FIG. 1A shows an image of a pupil and FIG. 1B shows an image of wavefront measurements of the eye of FIG. 1A using Hartmann-Shack sensor for the post-op PRK eye. The post-op PRK eye has a visual acuity of 20/20 but experiences night vision symptoms of glare and ghost images. The wavefront sensor image in FIG. 1B indicates that the PRK procedure may have caused irregularity at the corneal surface, as indicated by irregularities of focus spot on the top. FIGS. 1C and 1D show images of the pupil and a Hartmann-Shack sensor generated image for the RK eye, respectively. The post-op RK eye has a visual acuity of 20/40, but experience glare, halo, and ghost image. Both of these surgical eyes (eyes having received refractive surgery) have serious night vision symptoms, which can create problems when driving at night.

With the development of new technologies for aberration-induced symptoms as disclosed in U.S. patent application Ser. No. 11/371,288, filed Mar. 8, 2006, and entitled Algorithms and Methods for Determining Aberration-Induced Vision Symptoms in the Eye from Wave Aberration by Liang, and which published as U.S. Patent Application Publication No. 2006/0203198 on Sep. 14, 2006, the disclosure of which is incorporated herein by reference in its entirety, vision symptoms of these surgical eyes can be diagnosed from the wavefront measurements. For example, a hypothetical retinal image of an acuity chart can be generated from measurements taken with a Hartman-Shack sensor, which generates an aberration wavefront map of an eye, using this technology.

FIG. 2A shows a wavefront map of the high-order aberrations in the post-op PRK eye for a pupil size of 4.8 mm in diameter. Low order aberrations, e.g., sphero-cylinder errors, such as defocus and astigmatism in the eye have already been removed before generating the map because they can be corrected by conventional glasses. FIG. 2B shows the calculated retinal images of an acuity chart. Even though the letters in the acuity charts are blurred, they could be still recognized. Aberration-induced symptoms of the post-op PRK eye are calculated using the Algorithms described in U.S. Patent Application Publication No. 2006/0203198, and shown in FIG. 2C for ghost images (faint arrows at the lower right of the bright arrows), and in FIG. 2D for aberration-induced glare at four to five o'clock directions. Night vision symptoms (ghost images and aberration-induced glare) in FIG. 2C and FIG. 2D were confirmed by the subjective experiences of the PRK patient.

FIG. 3A shows a wavefront map of the high-order aberrations in the post-op RK eye for a 4.2 mm in diameter. Defocus and astigmatism in the eye have already been removed because they can be corrected by conventional glasses. FIG. 3B shows the calculated retinal images of an acuity chart. It is clearly seen that the eye can barely recognize the acuity letter because of image blurs caused by uncorrected high-order aberrations. Aberration-induced symptoms are shown in FIG. 3B for ghost images (below the bright arrows), and significant aberration-induced glare (strong from 5 to 7 o'clock directions and weaker but everywhere in the view) in FIG. 3D. Because of the night vision symptoms (glare, ghost images), the post-op RK patient cannot drive at night.

Symptoms in the surgical eyes shown in FIGS. 2A-D and 3A-D illustrate vision problems common to hundreds of thousands of people having had refractive surgery. Even though these surgical eyes have acceptable visual acuity of 20/40 or better, they are suffering from various vision symptoms that have never been diagnosed clinically. Refractive surgery induced night vision symptoms can make night driving a dangerous task or impossible for many post-op patients.

More importantly, surgical eyes with symptoms shown in FIG. 2B-D and FIGS. 3B-D are not treatable with conventional refractive corrections. First, vision of these surgical eyes cannot be restored by additional laser vision correction because damages were made in the corneal surfaces. Second, aberrations in these surgical eyes are not correctable by conventional sphero-cylindrical corrections using refractive glasses, contact lenses and intraocular lenses. Generally, the only possibility to fix these surgical eyes typically is a corneal transplant, but corneal transplantation is a complex procedure with potentially unpredictable outcomes.

Regarding vision correction devices, impact of a correction lens on the light wave entering an eye can be described by a complex function U(x,y) as
U(x,y)=a(x,y)exp(−2πiW_{c}(x·y)/λ) [1]

where a(x,y) is a real function describing the amplitude light transmittance function across pupil of the eye, and W_{c}(x,y) describes refractive corrections in term of wavefront distribution across the pupil, and λ is the wavelength of light wave.

If a(x,y)=1, the correction lens does not change total photon energy for the retinal image. If a(x,y) has an intensity profile or is less than 1, the correction lens reduces total photon energy into the eye. Energy efficiency across the pupil is represented by an intensity light transmittance function A(x,y) that equals to |a(x,y)|².

Up until now, vision corrections typically have been by the so-called sphero-cylindrical correction approach that corrects for focus error and cylindrical error in the eye, and has a uniform light transmittance across the pupil (a(x,y)=1). Even though effective with spectacles, contact lenses and intro-ocular lenses, conventional sphero-cylindrical corrections have a number of limitations. First, night vision can be poor for eyes with significant high-order aberrations that are not corrected in conventional corrections. Night vision problems are often described as glare, halo, ghost image and starburst. Second, most eyes can be corrected for a visual acuity of 20/40 or better while only a small portion of eyes can be corrected for an acuity of 20/10.

Lenses with Controlled Pupil Transmittance (CPT) were first proposed more than 40 years ago. Various types of lenses with CPT include: (1) contact lenses with controlled pupil size as disclosed for cosmetic and therapeutic enhancements in U.S. Pat. No. 3,536,386, entitled Contact Lenses with Simulated Iris, filed on Oct. 27, 1967 by Spivack; (2) contact lenses with annular mask as disclosed for presbyopic corrections in U.S. Pat. No. 5,245,367, entitled Annular Mask Contact Lenses, filed on Nov. 12, 1991 by Miller, et al.; (3) contact lenses with controlled transmittance profiles as disclosed in U.S. Pat. No. 4,576,453, entitled Light-Occluding Contact Lens, filed on Aug. 3, 1984 by Borowsky, and in U.S. Pat. No. 5,905,561, entitled Annular Mask Lens Having Diffraction Reducing Edges, filed on Jun. 14, 1996 by Lee et al.

It is not surprising to find that these lenses with controlled pupil transmittance (CPT) have not found any clinical acceptance yet because successful commercialization of lenses with CPT must overcome a number of fundamental obstacles. First, all lenses with CPT reduce total light into the eye and impacts of reduced retinal luminance has not been properly studied.

There is a need to develop a method to quantitatively describe the loss in retinal intensity and retinal brightness for a lens with CPT to ensure that the loss in photon is acceptable for night vision. There also is a need for appropriate means to ensure that these lenses with CPT can produce measurable gains in image quality. Gains in night vision must be measurable to justify potential losses in retinal brightness and retinal intensity. Gains can be specified in terms of visual acuity, vision clarity, or night symptoms. But there is no known method in the prior art is capable of these complicated tasks. Third, methods must be developed to ensure that gains in image quality can offset losses in light efficiency for night vision so that an intelligent design can be achieved. There also is a need for an effective clinical procedure to measure gains and losses by lenses with controlled pupil transmittance (CPT) in reference to conventional lenses. Lenses with CPT require a high-level customization for individual eyes because high-order aberrations are different from eye to eye.

Other things that can impair night vision include color contact lenses and intraocular lenses. Color contact lenses are commercially available in two general categories: tinted color contact lenses and opaque color contact lenses. Acceptance of these color contact lenses is very limited (about 3%) partially because they do not work well for night vision or at low-light conditions.

Commercial opaque color contact lenses usually comprise a non-opaque pupil section with a diameter of about 5 mm, an iris section with at least a colored, opaque intermittent pattern that leaves a substantial portion within the interstices of the pattern non-opaque. In a well-lit situation, natural pupil of an eye is often relatively small (about 4 mm) and the natural iris of the eye will overlap with the iris section of the opaque color contact lens such that the appearance of the eye's iris is altered or enhanced by blending the color pattern in the iris section of the contact lens with the natural iris of the eye through the non-opaque portion of the color opaque contact lens. In a low-lit situation or for night vision, however, natural pupil of an eye is often large (around 7 mm) and light energy will pass trough the non-opaque portion of the iris section of the contact lens and reach the retina of the eye as scattering light or diffraction light. Therefore, conventional opaque color contact lenses are not appropriate for wearing at night because they can cause vision symptoms like ghost images, halos, or glare for night vision.

Conventional tinted color contact lenses are made by dispersing a dye throughout a lens. Light is slightly reflected by the tinted lenses to create a desired color addition to the natural color of the iris of an eye. Conventional tinted lenses are limited for at least two reasons. First, tinted lenses can only alter iris color slightly for eyes with light iris because reflectivity of the tinted lenses cannot be too high (often less than 20%). Otherwise, tinted contact lenses with high reflectivity will have problems for low-light vision because of reduced luminance efficiency. Second, tinted color contact lenses are not favored for night vision because they reduce total light into the eye and do not reduce contribution of image blur caused by high-order aberrations.

Although methods for making other cosmetic and therapeutic contact lenses such as a contact lens with a restricted pupil sizes were disclosed in U.S. Pat. No. 3,536,386, which issued to Spivack more than 30 years ago, clinical practice with Spivack's lenses is believed not to have been possible for at least three reasons. First, it is generally clinically impractical to prescribe a contact lens with a restricted pupil size if all the determining factors like image intensity, image quality, field of view, and visual acuity must be measured and compared clinically for different pupil sizes. Second, it has been generally believed that improving retinal image quality by restricting natural pupil size of a normal eye is at the expense of reduced retinal intensity. Reducing retinal intensity for night vision is fundamentally negative for night vision performance. Third, the pupil size for the best retinal image quality is about 3 mm for an average eye and a contact lens with a restricted pupil size of 3 mm for normal human eyes is generally not acceptable because of a low light efficiency and a reduced field of view.

Implantable ophthalmic lenses, which also are referred to as intraocular lenses (IOLs), fall into three basic catagories: intraocular lenses for cataract suregery, intraocular contact lenses placed behind the iris and in front of the crystalline lens purely for refractive correction, and Phakic intraocular lenses implanted between the cornea and the iris of an eye for refractive correction. The optic for all conventional implantable ophthalmic lenses generally is a lens having about a 5 mm to 7 mm diameter with a its transparency shown in the light transmittance profile depicted in FIG. 34A.

FIGS. 34B-D illustrate known intraocular lens designs. FIG. 34B shows a typical intraocular lens comprising an optic having an optical lens and haptics to fixate the lens inside the eye. Rigid intraocular lenses are usually inexpensive, but generally not favored in cataract surgeries because they require a large incision and stitches. However, new materials such as acrylic and silicone have been used to make intraocluar lenses foldable so that intraocular lenses having diameters of 5 mm to 7 mm can be folded and inserted into an eye through a smaller incision about 3 mm without using stitches.

Even though cataract surgery is a mature procedure and performed routinely, a person's vision after receiving a conventional intraocular lenses is not always trouble-free, particularily at night when the person's pupil size is relatively large as compared to it size in daylight. Among the concerns with an intraouclar lens include a decentered lens implanted in an eye, light scattering by the edge of a rigid intraocular lens specially designed for a small incision, and light scattering by a portion of the haptics at night when the pupil of a person's eye is relatively large. Further, replacing the natural lens of an eye with a man-made lens can increase high-order aberrations of the eye and cause degraded night vision for many people.

People who have had cataract surgery and have received an intraocular lens typically wear spectacles or contact lenses because most intraocular lenses do not have the capability to accommodate focus power at different distances. Post catatract surgery eyes are often designed to provide good distance vision without refractive correction, but require the patient to wear reading glasses for near vision. FIG. 34C shows a multifocal intraocular lens comprising a multifocal lens and haptics to fixate the lens inside the eye. Unlike a conventional intraocular lens of a single focal length as shown in FIG. 34B, the optical lens for the multifocal lens has multiple optical zones to achieve an acceptable correction for both near and far vision. Despite its success in achieving acceptable acuity for near and far vision, multifocal intraocular lenses often increase chances for night vision symptoms like glare, halo and ghost images. Therefore, even though a person having a multifocal intraocular lens can pass driver's vision tests and read text without any refractive correction, driving at night with a multifocal lens can be problematic due to potential night vision symptoms.

An alternative to the multifocal lens is a psedu-accommodation intraocular lens, shown in FIG. 34D. The illustrative accommodation intraocular lens has a unitary optic, haptics to fixate the lens to the eye, and a hinge like a V-grove in each haptic arm to make each haptic arm flexible. The optic of an accommodation intraocular lens is either a rigid lens or a semi-flexible lens. If a rigid lens is used, the focal length of an eye can be adjusted by moving the lens forward or backward with the hinge when the eye tries to accommodate at different view distance. If a semi-flexible lens is used, focal length of an eye can be adjusted by deforming the lens shape or by moving the lens forward or backward when the eye try to accommodate at different view distance. The design in FIG. 34D is innovative, but also has some practical concerns. The optical lens cannot be too big to be implanted through a small incision because the lens is either a rigid lens or a semi-flexible lens. A large lens also is hard to move to effect the accomodation because of its large surface. If the accommodation is achieved through deformation of a semi-flexible lens, a large lens requires a strong force to deform and can create additional high-order aberrations. Further, the lens cannot be too small or it may cause night vision symptoms.

In light of the forgoing, there is a need to provide practical means to improve night vision of human eyes, in particular for eyes that have surgically induced night vision symptoms.

### SUMMARY OF THE INVENTION

The present invention involves methods and apparatus to improve vision.

According to one embodiment of the invention, a method of selecting an ophthalmic device to improve night vision comprises obtaining a wave aberration of the pupil of a patient's eye using wavefront analysis; and selecting a transmittance profile for at least a portion of the device to control light transmittance through the pupil of the eye.

According to another embodiment of the invention, a method of prescribing an ophthalmic device with controlled optical light transmittance to improve night vision comprises obtaining a wave aberration data of a patient's eye; determining the uncorrected aberrations of the eye by removing predetermined aberrations; selecting a plurality of light transmittance profiles for the device; calculating optical quality of the eye using complex pupil functions from the determined uncorrected aberrations and the selected light transmittance profile; determining a light transmittance profile from the selected light transmittance profiles by optimizing vision between retinal image quality and retinal image intensity; providing a prescription of an ophthalmic device including a specification of refractive correction if the patient is myopic or hyperopic and the determined light transmittance profile.

According to another embodiment of the invention, a method for determining a light transmittance profile of an ophthalmic device for improving night vision of human eyes comprises obtaining a wave aberration of an eye and a manifest refraction if the eye is myopic and hyperopic; determining the uncorrected aberrations in the eye by removing certain aberrations in the eye; calculating optical quality of an eye based on the determined uncorrected aberrations; finding the best corrected optical quality of the eye such as the best MTF in all possible pupil size in a natural pupil and the optical quality of the eye with the larges natural pupil at night without controlling pupil light transmittance; determining a light transmittance profile for the device that offers an improved night vision quality that ranks between the best corrected optical quality in all possible pupil sizes and the optical quality with the largest natural pupil at night without controlling pupil light transmittance.

According to another embodiment of the invention, a method of prescribing an ophthalmic device with controlled optical light transmittance for improving human vision comprises obtaining a manifest refraction of an eye, measuring optical quality of the eye with a plurality of pupil light transmittances; determining a light transmittance profile by optimizing vision between retinal image quality and retinal image intensity; and providing a prescription of an ophthalmic device that contain a specification of light transmittance and refractive correction.

According to another embodiment of the invention, an ophthalmic device for improving night vision comprises a disk shaped member having a clear central optical portion with a diameter that is custom determined based on wave aberrations of a patient's eye and an outer annular portion surrounding the central optical portion and having reduced light transmittance as compared to the clear central optical portion.

According to another embodiment of the invention, an ophthalmic device for improving night vision comprises a disk shaped member having a clear central optical portion and an annular portion surrounding the central optical portion, the central optical portion having an outer diameter from 3.25-5.5 mm, the annular portion having an outer diameter of 3.75 to 8.75 mm and comprising material that provides light transmittance of 5-50% of visible light to pass therethrough.

According to another embodiment of the invention, an intraocular lens comprises an optic portion and at least one haptic, the optic portion consisting of a central clear optical section adapted to focus light toward a retina of an eye and a an annular section comprising material having properties such that the annular section transmittance is 5-50%.

According to another embodiment of the invention, an intraocular lens comprises at least one haptic; an optic comprising a central optical clear section and an annular opaque section, the central optical clear section having an outer diameter of 3.3 mm to 4.5 mm and being adapted to focus light toward a retina of an eye, the annular opaque section surrounding the clear section to block photons of visible light from passing therethrough the central clear optical section.

According to another embodiment of the invention, a corneal inlay comprises and annular member configured and sized for implantation in a cornea of a human patient, the annular member having an anterior face and a posterior face and a plurality of hole pairs, each hole pair having a first hole partially extending into the annular member from the anterior face and a second hole partially extending into the annular member form the posterior face, the annular member having a plurality of channels formed therein, each channel fluidly coupling a hole pair.

The above is a brief description of some deficiencies in the prior art and advantages of the present invention. Other features, advantages, and embodiments of the invention will be apparent to those skilled in the art from the following description and accompanying drawings, wherein, for purposes of illustration only, specific forms of the invention are set forth in detail.

### BRIEF DESCRIPTION OF THE DRAWINGS

FIGS. 1A-D are wavefront measurements made with a Hartmann-Shack wavefront sensor for two surgical eyes, where FIG. 1A depicts an image of a post-PRK eye, FIG. 1B depicts the wavefront image from a Hartmann-Shack sensor of the eye in FIG. 1A, FIG. 1C depicts an image of a post-RK eye and FIG. 1D depicts the wavefront image from a Hartmann-Shack sensor of the eye in FIG. 1C.

FIGS. 2A-2D illustrate a wavefront map as well as the calculated retinal images for the eye shown in FIG. 1A-B where FIG. 2A shows the wavefront map for a pupil size of 4.8 mm in diameter, FIG. 2B-D show calculated retinal images of an eye after a PRK procedure and the retinal images are obtained by convolving specially-designed objects with a point-spread function of the eye derived from obtained wave aberrations of the eye for night vision and more particularly FIG. 2B illustrates a blurred acuity chart, FIG. 2C illustrates ghost images of an arrow caused by high-order aberrations that the PRK induced, and FIG. 2D illustrates night glare of an extended light source to which the PRK procedure caused or contributed.

FIG. 3A-3D illustrate a wavefront map as well as the calculated retinal images for the eye shown in FIG. 1C-D where FIG. 3A shows the wavefront map for a pupil size of 4.2 mm in diameter FIG. 3B-D show calculated retinal images of an eye after an RK procedure and the retinal images are obtained by convolving specially-designed objects with a point-spread function of the eye derived from obtained wave aberrations of the eye for night vision and more particularly FIG. 3B illustrates a blurred acuity chart, and FIG. 3C illustrates ghost images of an arrow caused by high-order aberrations induced by the RK procedure, and FIG. 3D illustrates night glare of an extended light source to which the PRK procedure caused or contributed.

FIGS. 4A-4E illustrate ophthalmic devices for therapeutic correction of night vision symptoms according to principles of present invention, where FIG. 4A shows the ophthalmic device with a clear central portion and an outer section for reduced light transmittance of light at pupil periphery according to the invention, FIG. 4B diagrammatically illustrates a contact lens embodiment in situ focusing light beams to create an image, FIG. 4C diagrammatically illustrates a contact lens embodiment according to the invention in situ; FIG. 4D illustrates an intraocular lens in situ according to another embodiment of the invention; and FIG. 4E illustrates another intraocular lens in situ according to another embodiment of the invention.

FIGS. 5A-C illustrate the use of a contact lens with controlled pupil light transmittance in FIG. 4B for therapeutic correction of eyes after refractive surgeries shown in FIG. 2A and FIG. 3A, where FIG. 5A diagrammatically illustrates a contact lens embodiment in situ focusing light beams to create an image, FIG. 5B illustrates the wavefront errors with a conventional correction (4.8 mm in diameter) and with a lens having controlled light transmittance (truncated within the dotted region), and 5C illustrates the wavefront errors with a conventional correction (4.2 mm in diameter) and with a lens having controlled light transmittance (truncated within the dotted region).

FIG. 6A-D show the wavefront errors as well as the calculated retinal images with a reduced effective pupil size where FIGS. 6B-D show the calculated retinal image of an eye after a PRK procedure and the retinal images are obtained by convolving specially-designed objects with a point-spread function of the eye derived from the truncated wave aberrations of the eye for night vision and more particularly FIG. 6B illustrates a relatively clear acuity chart, FIG. 6C illustrates the elimination of ghost images of an arrow caused by high-order aberrations induced by the PRK procedure, and FIG. 6D illustrates the elimination of night glare of an extended light source to which the PRK procedure caused or contributed.

FIG. 7A-D show the wavefront errors as well as the calculated retinal images with a reduced effective pupil size where FIGS. 7B-D show the calculated retinal image of an eye after a RK procedure with a wavefront-controlled pupil transmittance according to the invention and the retinal images are obtained by convolving specially-designed objects with a point-spread function of the eye derived from the truncated wave aberrations of the eye for night vision and more particularly FIG. 7B illustrates a relatively clear acuity chart, FIG. 7C illustrates the elimination of ghost images of an arrow caused by high-order aberrations that the RK procedure induced, and FIG. 7D illustrates the elimination of night glare of an extended light source to which the PRK procedure caused or contributed.

FIG. 8 is a block diagram illustrating methods and systems for designing and prescribing a therapeutic lens according to one embodiment of the invention for reducing and/or eliminating night vision symptoms such as glare, halo, and/or ghost images.

FIG. 9 illustrates the retinal image quality (Modulation Transfer function) of normal eye with a visual acuity of 20/20 or better and under a conventional sphero-cylindrical correction for best vision and night vision.

FIG. 10 is a flow chart illustrating a method for optimizing vision performance of an eye using a device with wavefront-optimized pupil transmittance in accordance to one embodiment of the present invention.

FIG. 11 is a graphic representation of retinal image quality (MTF volume) under two approaches for controlling pupil transmittance.

FIG. 12 is a graphic illustration depicting retinal image quality, retinal intensity and retinal brightness of averaged normal human eyes for a plurality of pupil sizes.

FIGS. 13A-H illustrate the retinal point-spread images of an eye for 4 different pupil sizes. The retinal images are normalized in two different approaches. FIGS. 13A, 13C, 13E, and 13G show the retinal point-spread functions for a pupil diameter of 2 mm, 4 mm, 6 mm, and 7.7 mm normalized to the peak intensity of an aberration-free eye at the same pupil size, respectively. FIGS. 13B, 13D, 13F, and 13H show the retinal point-spread functions for a pupil diameter of 2 mm, 4 mm, 6 mm, and 7.7 mm scaled according to the total energy for each pupil dimension, respectively.

FIGS. 14A-14F illustrate retinal images of an acuity chart of an aberration-free eye as well as two real eyes. Two pupil sizes are considered for the calculations. FIG. 14A and FIG. 14B show retinal images of an acuity chart of an aberration-free eye for a pupil size of 6 mm and for a pupil size of 3 mm, respectively. Retinal images for two real eyes of different persons are also shown for a pupil size of 6.7 mm pupil (FIG. 14C, FIG. 14E) and for a 3.75 mm pupil (FIG. 14D, and FIG. 14F).

FIG. 15 is a flow chart depicts optimizing vision performance of an eye using a refractive correction with controlled pupil transmittance according to another embodiment of the invention.

FIGS. 16A-F illustrate the calculated retinal images for an eye under conventional correction without controlling pupil transmittance (FIG. 16A, FIG. 16C, and FIG. 16E) and those with controlled pupil transmittance (FIG. 16B, FIG. 16D, and FIG. 16F) according to one embodiment of the invention.

FIGS. 17A-F illustrate the calculated retinal images for another eye under conventional correction without controlling pupil transmittance (FIG. 17A, FIG. 17C, and FIG. 17E) and those with controlled pupil transmittance (FIG. 17B, FIG. 17D, and FIG. 17F) according to one embodiment of the invention.

FIG. 18 is a flow chart depicting a procedure for estimating visual acuity of eye from wave aberration of an eye according to one embodiment of the invention

FIGS. 19A-F illustrate retinal image of an acuity chart for three out of 21 eyes that show no or little improvement by the optimized pupil transmittance according to one embodiment of the invention. Images of three eyes under a conventional correction without controlling pupil transmittance are shown in FIG. 19A, FIG. 19C, and FIG. 19E, while images of the same three eyes under a correction with controlled pupil transmittance are shown in FIG. 19B, FIG. 19D, and FIG. 19F according to one embodiment of the invention.

FIGS. 20A-F illustrate retinal image of an acuity chart for three out of 21 eyes that shows improvements in vision clarity by the optimized pupil transmittance according to one embodiment of the invention. Images of three eyes under a conventional correction without controlling pupil transmittance are shown in FIG. 20A, FIG. 20C, and FIG. 20E, while images of the same three eyes under a correction with controlled pupil transmittance are shown in FIG. 20B, FIG. 20D, and FIG. 20F according to one embodiment of the invention.

FIGS. 21A-R illustrate retinal image of an acuity chart for nine out of 21 eyes that shows improvements in vision acuity by one line with the optimized pupil transmittance according to one embodiment of the invention. Images of 9 eyes under a conventional correction without controlling pupil transmittance are shown in FIG. 21A, FIG. 21C, FIG. 21E, 21G, FIG. 211, FIG. 21K, FIG. 21M, 21O, and FIG. 21Q, while images of the same 9 eyes under a correction with controlled pupil transmittance are shown in FIG. 21B, FIG. 21D, FIG. 21F, 21H, FIG. 21J, FIG. 21L, FIG. 21N, 21P, and FIG. 21R according to one embodiment of the invention.

FIGS. 22A-J illustrate retinal image of an acuity chart for five out of 21 eyes that shows improvements in vision acuity by more than one line with the optimized pupil transmittance according to one embodiment of the invention. Images of three eyes under a conventional correction without controlling pupil transmittance are shown in FIG. 22A, FIG. 22C, FIG. 22E, 22G, FIG. 22I while images of the same 5 eyes under a correction with controlled pupil transmittance are shown in FIG. 22B, FIG. 22D, FIG. 22F, FIG. 22H, FIG. 22J according to one embodiment of the invention.

FIG. 23 is a flow chart illustrating a method for prescribing a refractive correction with controlled pupil transmittance through clinical refraction of an eye for a plurality of pupil sizes

FIG. 24 is a flow chart illustrating a wavefront-assisted manifest refraction.

FIGS. 25A-D show point-spread function of a normal human eye at four different natural pupil sizes, where FIG. 25A corresponds to one pupil size of 2 mm, FIG. 25B corresponds to a second pupil size of 4 mm, FIG. 25C corresponds to a third pupil size of 6 mm, and FIG. 4 corresponds to a fifth pupil size of 7.7 mm.

FIG. 26 is a graphic illustration showing integrated intensity of a point object for an average normal human eye as well as for an ideal aberration-free eye. The integrated intensity is the total energy that is centered at the peak of the point-spread function over a retina patch of 1 arc minute, 3 arc minutes, and 5 arc minutes, respectively.

FIG. 27 shows the transmittance profile of a partially opaque lens with improved night vision according to another embodiment of the invention.

FIG. 28 shows the transmittance profile of a partially attenuated lens, which can be worn on or implanted in an eye, with improved night vision according to another embodiment of the invention.

FIG. 29A is a front view of an opaque color lens, which can be worn on or implanted in an eye, with improved night vision accord according to another embodiment of the present invention.

FIG. 29B is a front view of another embodiment of an ophthalmic device, which can implanted in the cornea of an eye, with improved night vision according to another embodiment of the present invention.

FIG. 29C is a sectional view of the device of FIG. 29B taken along line 29C-29C.

FIG. 29D illustrates a variation of the configuration shown in FIG. 29C.

FIG. 30 is a front view of a black, opaque lens with improved night vision according to another embodiment of the invention.

FIG. 31 is a front view of a tinted color lens with improved night vision according to another embodiment of the invention.

FIG. 32A is a front view of a transition lens with improved night vision in a first state according to another embodiment of the invention.

FIG. 32B is a front view of the lens of FIG. 32A in a second state.

FIG. 33 is a flow chart illustrating a process for selecting an ophthalmic device with controlled pupil transmittance in accordance to the present invention.

FIGS. 34A-D illustrate traditional intraocular lenses where FIG. 34A shows a uniform light transmittance profile that applies to each of the lenses, FIG. 34B depicts a traditional mono-focal intraocular lens, FIG. 4C depicts a traditional multifocal intraocular lens, and FIG. 4D illustrates a conventional accommodation intraocular lens.

FIG. 35A is a front view of a tinted intraocular lens embodiment according to the invention.

FIG. 35B shows the transmittance profile of the tinted intraocular lens of FIG. 35A.

FIG. 35C is a front view of another tinted intraocular lens embodiment according to the invention.

FIG. 35D shows the transmittance profile of the tinted intraocular lens of FIG. 35C.

FIG. 36A1-3 show another intraocular lens embodiment according to the invention, where FIG. 36A-1 shows the entire device, FIG. 36B-2 shows the clear lens of the device, and FIG. 36A-3 shows the annular reduced transmittance portion of the device.

FIG. 37A1-3 show another intraocular lens embodiment according to the invention with accommodation control, where FIG. 37A-1 shows the entire device, FIG. 37B-2 shows the clear lens of the device, and FIG. 37A-3 shows the annular reduced transmittance portion of the device.

FIG. 38A is a front view of another tinted intraocular lens embodiment according to the invention.

FIG. 38B shows the transmittance profile of the tinted intraocular lens of FIG. 38A.

FIG. 39A is a front view of another tinted intraocular lens embodiment according to the invention.

FIG. 39B shows the transmittance profile of the tinted intraocular lens of FIG. 39A.

FIG. 40 is a flow chart depicting a process for designing a customized intraocular lens with controlled pupil transmittance.

FIG. 41 is a diagrammatic representation of variation in pupil size at different distances from the corneal first surface.

### DETAILED DESCRIPTION OF THE INVENTION

Before the present invention is described, it is to be understood that this invention is not intended to be limited to particular embodiments or examples described, as such may, of course, vary. Further, when referring to the drawings like numerals indicate like elements. And when describing dimensions or values, “is X-Y” or “is X to Y” or “of X-Y” or “of X to Y” means one or more values selected from X, Y and any value therebetween.

According to one embodiment of the invention, light transmittance (hereinafter referred to as transmittance) of light through a pupil or to the retina of a patient's eye is controlled to improve night vision. In one example, this involves providing an ocular device in the form of a contact lens having a central, disk-shaped, clear window having a diameter that is custom selected based on eye's wave aberration and less than the diameter of the patient's pupil at night (i.e., the diameter of the patient's pupil when subjected to low-lit conditions). Therefore, if a patient's pupil enlarges to a maximum size with a diameter of 8 mm when subjected to low-lit situations, the center window will be less than 5.5 mm. The ocular device also has an annular portion that surrounds the disk shaped window portion. The annular portion comprises material that provides reduced light transmittance. Although reducing the amount of light that reaches the retina at night is contrary to conventional wisdom, I have found that a controlled reduction of light or controlled light transmittance to the retina of a patient with pupil aberrations, e.g., controlled light transmittance across the pupil, can (1) reduce photon noise that otherwise can exacerbate halos and ghosts that one can experience at night and/or (2) improve night vision contrast with minimal reduction in retinal intensity. In the case of a contact lens, light transmittance is controlled across the corneal. In the case of an Intraocular lens, light transmittance is controlled across the pupil. And in the case of a corneal inlay, light transmittance is controlled within the corneal of an eye.

Although one ocular device is described for treating a patient's eye, another device can be provided to provide controlled transmittance of light through the pupil of the other eye of the patient.

Examples of methods for determining desired light transmittance profiles and constructions of ocular devices that provide a desired light transmittance profile will be described in more detail below.

Refractive Corrections with Enhanced Night Vision (ENV)

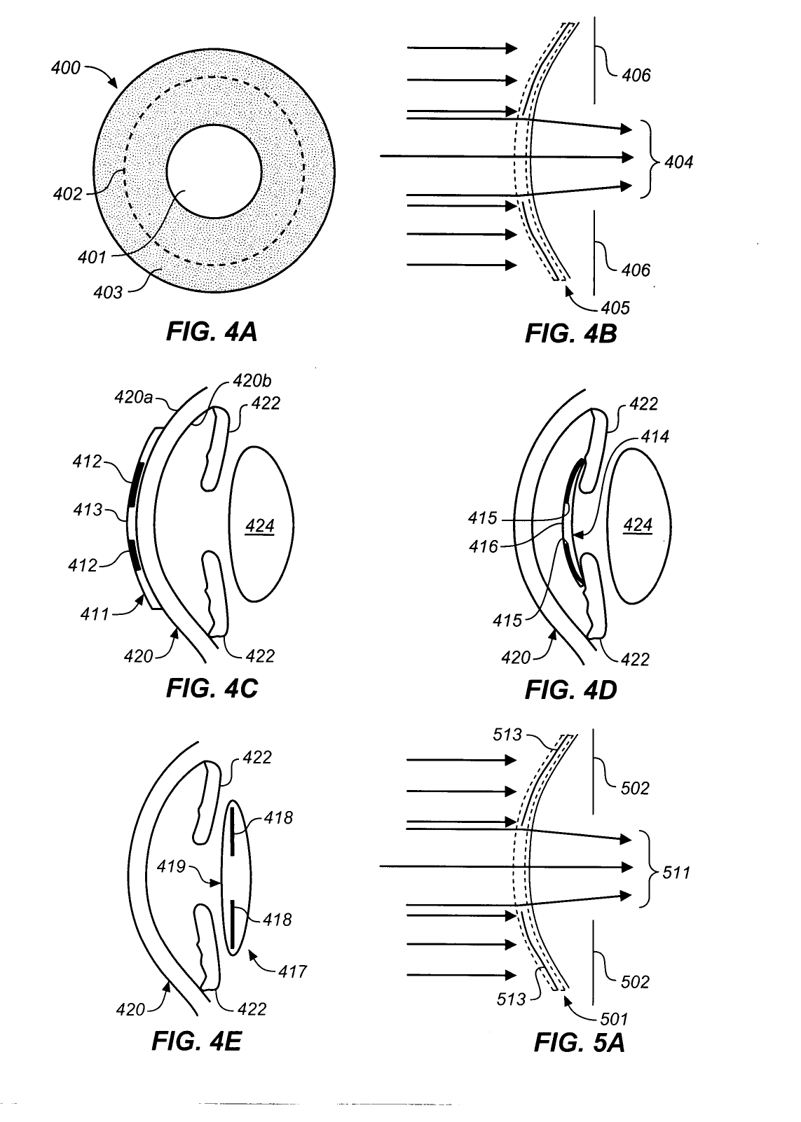

FIGS. 4A-E illustrate methods and devices for refractive corrections with ENV in according to several embodiments of invention. The device comprises an optical element for selecting a portion of optics within the natural pupil of an eye. In the embodiment shown in FIG. 4A, an optical element 400 contains an inner transparent optical zone 401 that is smaller than the largest natural pupil 402 of the treated eye (i.e., the maximum pupil size described throughout the description is the maximum pupil size in the dark and without dilation solution and this size may be achieved at night) and an outer segment 403 that blocks or attenuates light beams therethrough and thus blocks or attenuates light from entering the eye beyond the allowed optical zone 401 and within the pupil periphery up to the largest natural pupil 402 at low-lit conditions.

As an illustration, FIG. 4B shows the impact of light at the corneal plane, when an ophthalmic device 405, which can be the same as device 400, is positioned on the anterior surface of the cornea. Device 405 allows light beams through the inner transparent optical zone for imaging light beams or rays 404 to pass therethrough and blocks or attenuates light beams or rays near the edge of the iris 406 so that high-order aberrations associated with a large natural pupil at night are removed from the retinal image. In this manner, vision problems associated with a large pupil size at night are minimized or eliminated.

Referring to FIGS. 4C-E, illustrative examples of vision correction devices to enhance night vision (ENV) are shown. FIG. 4C depicts a contact lens. FIG. 4D depicts a Phakic intraocular lens and FIG. 4E depicts an intraocular lens for cataract surgeries. The optics of an eye includes cornea 420, iris 422 and crystalline lens 424.

Referring to FIG. 4C, contact lens 411 with ENV comprises an inner transparent optical zone 413 that corrects for refractive errors such as defocus and astigmatism, if necessary. Otherwise transparent optical zone is selected so as not to provide refractive correction. Contact lens 411 also includes outer segment 412 that blocks or attenuates light beams beyond the optical zone 413 and within pupil periphery up to the largest natural pupil at low-lit conditions. Contact lens 411 with ENV provides optimized vision quality at night by removing unwanted high-order aberrations associated with a large pupil as will be described in more detail below. Inner zone or portion 413 generally has a disk shape and outer segment or portion 412 is generally annular and forms a portion of the lens that surrounds inner portion 413.

FIG. 4D shows a Phakic intraocular lens 414 in accordance with the present invention. The Phakic intraocular lens with ENV comprises a transparent optics 416 that corrects refractive errors of the eye such as defocus and astigmatism if necessary, and an outer segment 415 that blocks or attenuates light beams beyond the optical zone 416 and within pupil periphery at low-lit conditions. The Phakic intraocular lens with ENV provides optimized vision quality at night by removing unwanted high-order aberrations associated with a large pupil at night.

FIG. 4E shows an intraocular lens 417 in accordance with the present invention. The intraocular lens with ENV comprises a transparent optics 419 that corrects refractive errors of the eye such as defocus and astigmatism, and an outer segment 418 that blocks or attenuates light beams beyond the optical zone 419 and within pupil periphery at low-lit conditions. The intraocular lens with ENV provides optimized vision quality at night by removing unwanted high-order aberrations associated with a large pupil at night.

Parameters in the refractive corrections with ENV include: (1) the size of the transparent optical zone, (2) the refractive powers in the transparent zone if any, (3) the size and transmittivity of the outer segment, which must be large enough to block or attenuate light beams at pupil periphery up to the largest natural pupil completely. The size of the transparent zone as well as the refractive power in the transparent zone should be determined based on optical quality of individual eyes at a plurality of pupil sizes.

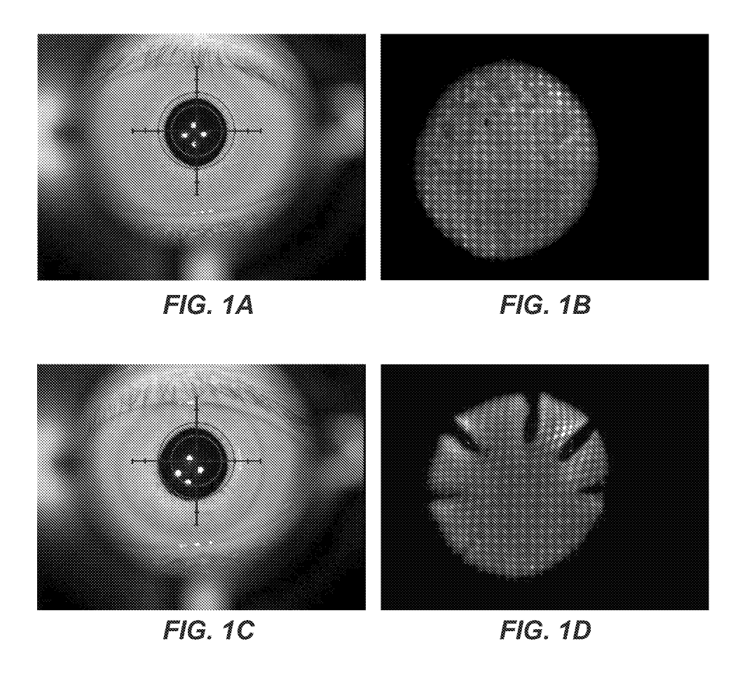

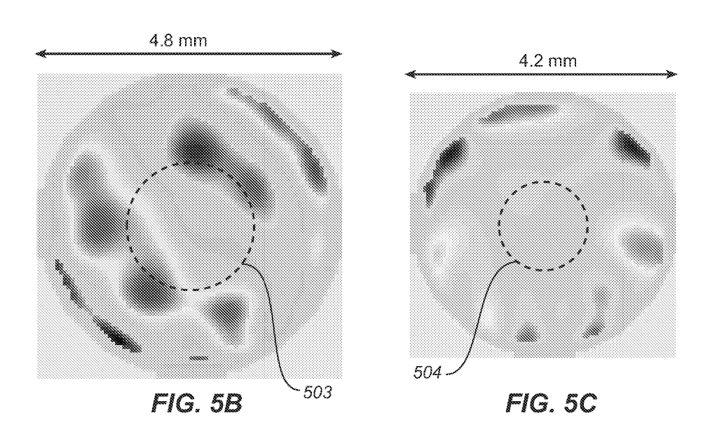

The ophthalmic devices illustrated in FIGS. 4A-E with ENV can address issues of night vision of eyes that have subject to a surgical procedure shown in FIG. 1 through FIG. 3. Referring to FIG. 5A, a designed refractive correction device is illustrated as a contact lens with ENV 501 in FIG. 5A same as the one in FIG. 4B. Contact lens 501 comprises an inner transparent optical zone 511 in the middle to correct defocus as well as astigmatism in the eye in a small pupil, and an outer segment 513 that blocks light beams beyond the inner transparent optical zone in pupil periphery up to the largest natural pupil at night.

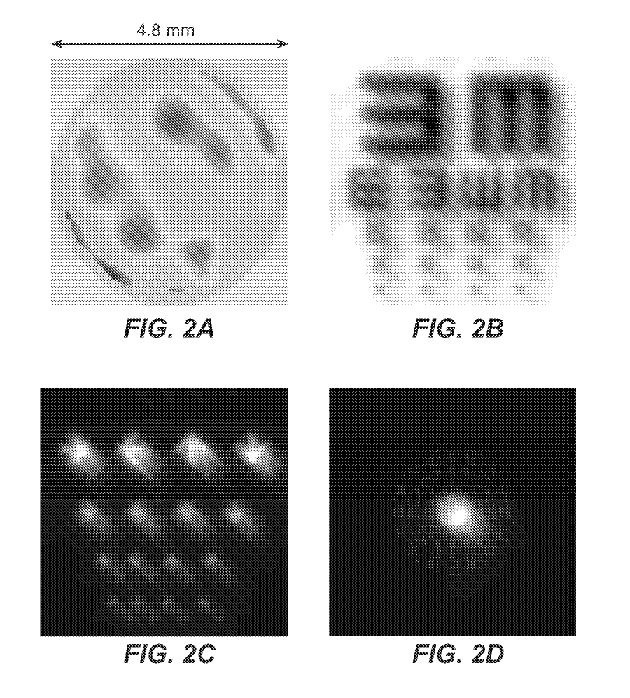

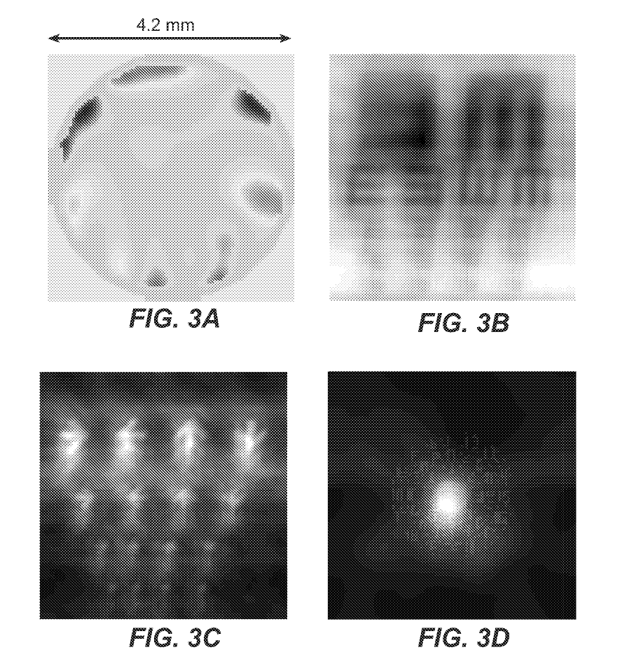

When such a vision correction is applied to the two surgical eyes having wavefront errors as shown in FIG. 2A and in FIG. 3A, wavefront errors associated with a large natural pupil 502 at night will be eliminated. For the post-op PRK eye shown in FIG. 5B, the diameter of the inner optical zone 503 is designed to be 2 mm while the natural pupil of the eye is larger than 4.8 mm, as shown in FIG. 5B. Wavefront errors within the largest natural pupil and beyond the inner optical zone (area outside the dotted circle) is removed from retinal imaging with the designed refractive correction. For the post-op RK eye as shown in FIG. 5C, the diameter of the inner optical zone 504 is 1.4 mm while the natural pupil of the eye is larger than 4.2 mm. Wavefront errors within the largest natural pupil and beyond the inner optical zone (area outside the dotted circle) are removed from retinal imaging with the designed refractive correction.

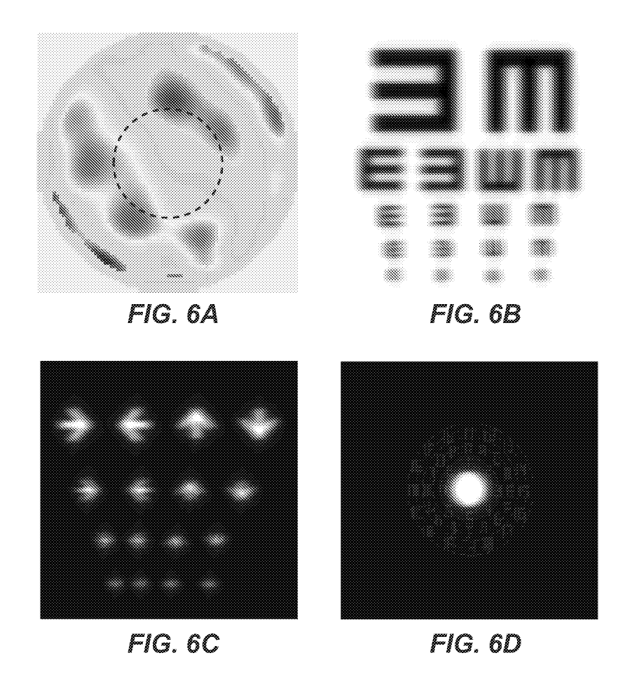

Referring to FIGS. 6A-D, wavefront error and retinal image of the post-op PRK eye under a refractive correction with ENV are shown. Referring to FIG. 5A and FIG. 6A, the correction element corrects sphero-cylindrical error of the eye within the dotted circle (2 mm in diameter) and blocks the wavefront error outside the dotted circle. FIG. 6B shows the retinal image of an acuity chart for the post-PRK eye using one of the ophthalmic devices described above (e.g., any one of devices 400, 405, 411, 414, 417, and 501). Letters in the acuity charts represent 20/80 (the largest letters), 20/40, 20/20, 20/15 and 20/10 (the smallest letters). It is clearly seen that the post-op PRK eye at night has a far better vision with the invention method shown in FIG. 6B than that with a conventional best sphero-cylindrical correction shown in FIG. 2B. Letters measured in 20/15 can be clearly seen under the refractive correction with ENV. More importantly, ghost images of the eye under a conventional vision correction in FIG. 2C are eliminated completely in FIG. 6C with the invention method. Glare around 4 to 5 o'clock in FIG. 2D under conventional correction is also eliminated completely in FIG. 6D with the invention method.

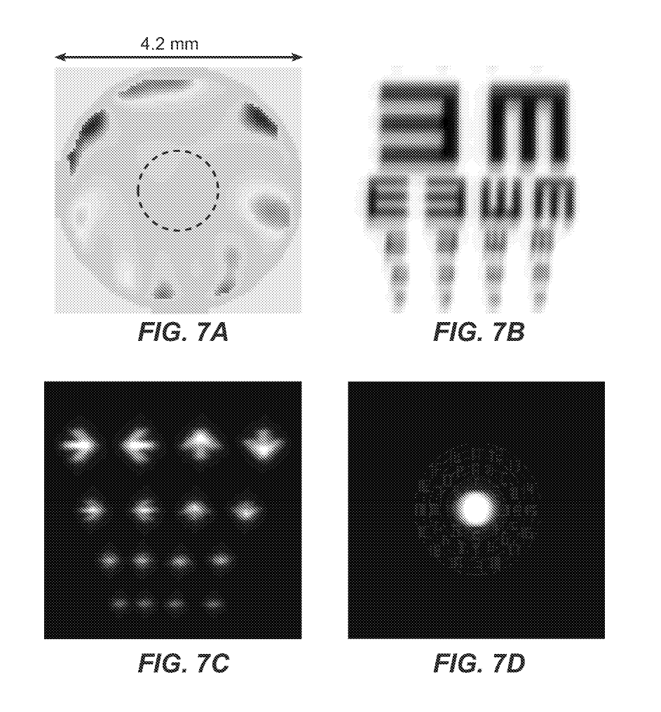

FIG. 7 shows wavefront error and retinal image of the post-op RK eye under the invention method. The correction element corrects sphero-cylindrical error of the eye within the dotted circle and blocks the wavefront error outside the dotted circle in FIG. 7A. FIG. 7B shows the retinal images of an acuity chart for the post-PRK eye with the invented correction. Letters in the acuity charts represent 20/80 (the largest letters), 20/40, 20/20, 20/15 and 20/10 (the smallest letters). It is clearly seen that the post-op RK eye at night has a far better vision with the invention method shown in FIG. 7B than that with a conventional best sphero-cylindrical correction shown in FIG. 3B. Letters measured in 20/40 and some in 20/20 can be clearly seen under the refractive correction with ENV. More importantly, vision symptoms of glare, ghost images of the eye under conventional vision in FIG. 3C and FIG. 3D are eliminated completely if the post-op RK eye is corrected with the invention method, shown in FIG. 7C and FIG. 7D.

Vision performances, depicted in FIGS. 6B-D and FIG. 7B-D, show that refractive corrections with Enhanced Night Vision (ENV) can address night vision symptoms in surgical eyes without corneal transplantation. By controlling the effective pupil sizes of the eye, the ENV technology improves image clarity for night vision, eliminates night symptoms such as ghost images and aberration-induced glare.

In cases of eyes that do not have a good optical zone in the middle of natural pupil, refractive corrections with ENV can select a portion of optics away from the pupil center if the natural pupil is large enough at night.

Methods and Systems for Enhancing Night Vision

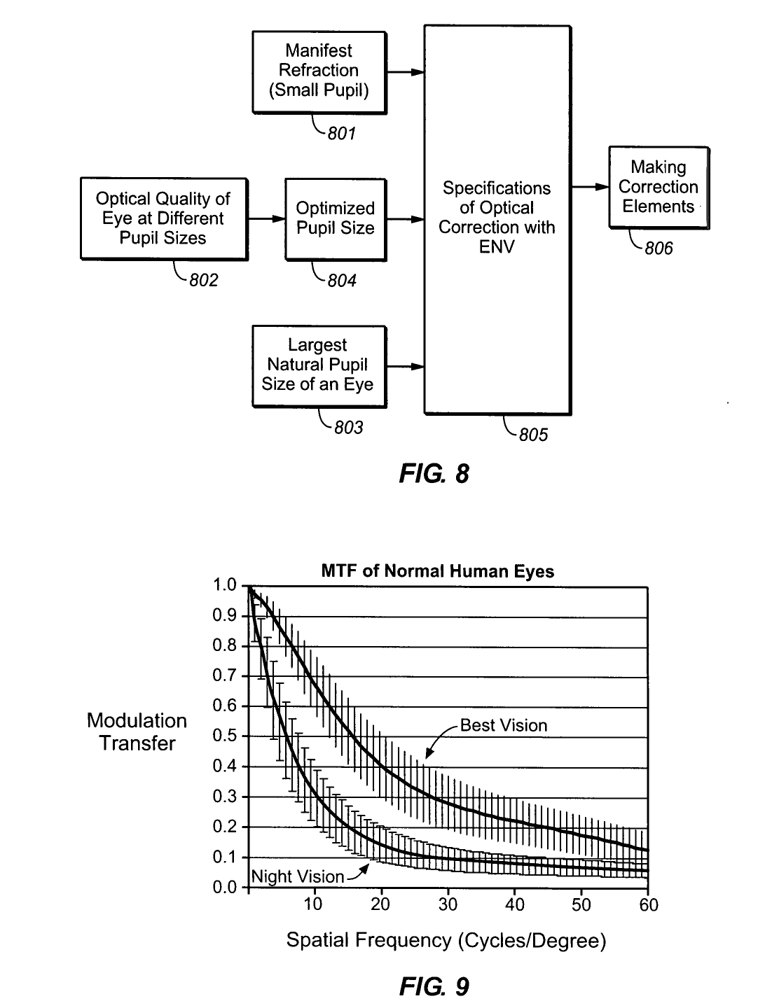

FIG. 8 shows methods and systems for achieving refractive corrections with ENV (Enhanced Night Vision). First, a subjective manifest refraction 801 for the sphero-cylindrical power is obtained for the eye using the convention refraction method in optometric practices, preferably with a relative small pupil between 1.5 mm and 4.5 mm. Second, optical quality of the eye is assessed at a plurality of pupil diameters 802, and an optimized pupil size 804 is determined for enhanced night vision. Third, natural pupil size of the eyes 803 is measured if the outer segment of refractive correction with ENV is different from eye to eye. Specifications of the refractive correction with ENV 805 include the size and the refractive correction of the inner transparent optical zone, and the largest natural pupil of the eye if necessary. Fourth, the ENV optical elements can be made as contact lenses, Phakic intraocular lenses and Intraocular lenses for cataracts. Embodiments of the outer segment in ENV may include a number of ways. It can be made by attaching or sandwiching an opaque layer into the convention correction lenses, or making the outer segment of the correction element using light absorbing materials.

Measuring optical quality at a plurality of pupil size can be achieved in a number of ways. It can be obtained by measuring wave aberration of eye using an aberrometers, including wavefront sensing with a Hartmann-Shack sensor. Optical quality of the eye as well as the best refractive correction can be derived from the measured wavefront for a plurality of pupil sizes. Wave aberration of an eye represents all aberrations in the eye including the low-order sphero-cylindrical error as well as high-order aberrations. The selected effective pupil area can be centered at the natural pupil or away from the center of the natural pupil depending on the distribution of the high-order aberrations. The optical quality of eye can be characterized with point-spread functions, modulation-transfer functions, calculated retinal images with an acuity chart, and calculated aberration-induced symptoms.

Optical quality of the treated eye at a plurality of pupil sizes can also be assessed by measuring modulation-transfer functions of the eye at different pupil sizes using double-pass measurement, known in the art. It can also be obtained by subjective refraction at a number of pupil diameters by an optician. In the case of using intraocular lenses for cataract eyes, corneal topography of the eye can be acquired for the determination of optical quality at a plurality of pupil sizes.

The optimized pupil sizes for ENV optics are determined for visual acuity for day vision and night vision, optimized vision clarity during the day and at night, and free from aberration-induced symptoms (ghost images, glare, and halo) at night. One way of optimization is to obtain the best optical quality (acuity, modulation transfer function) for all the possible pupil size for an eye.

Refractive Corrections with ENV for Normal Population

The invention methods of refractive correction with ENV can not only address vision symptoms for surgical eyes, but also improve night vision of normal eyes in normal population.

Wave aberrations for more than 200 normal human eyes (visual acuity 20/20 and better) without refractive correction were measured with a Hartmann-Shack sensor without pupil dilation. FIG. 9 shows the averaged Modulation-Transfer Functions (MTF) plus and minus one standard deviation for the cohort of normal human eyes when a conventional sphero-cylindrical correction is applied to the tested wavefront. MTFs for night vision were obtained from wave aberration of the eyes at the largest natural pupil ranging from 5 mm to 8 mm from eye to eye. The MTFs for best vision were obtained from wave aberration of the eyes by finding the best MTF of the tested eyes in all pupil sizes smaller than the measured natural pupil. The optimized pupil size for the cohort of normal eyes ranges from 1.5 mm to 4 mm from eye to eye.

Refractive corrections with ENV for the normal population can be achieved using the optimized pupil size for the inner transparent optical zone and configured in FIG. 4C for contact lenses, in FIG. 4D for Phakic intraocular lenses, and in FIG. 4E for an intraocular lens in cataract surgeries. Vision of normal population at night is improved from the lower MTF curve shown in FIG. 9 with the conventional sphero-cylindrical corrections to the top MTF curve shown in FIG. 9 with ENV.

When the optimized pupil size for ENV is small and less than 3 mm in diameter, compromises may be made to balance the optical quality and total light level for night vision.

Lenses with Wavefront-Optimized Pupil Transmittance

Conventional lenses correct for the focus error and the cylindrical error in the eye without changing light transmittance across pupil of the eye. Lenses with CPT can further improve vision beyond a sphero-cylindrical correction by attenuating or blocking light at periphery pupil. In order to gain control of a predictable retinal image quality, wave aberration in the eye has to be factored in so that impacts of controlled transmittance can be assessed for individual eyes.

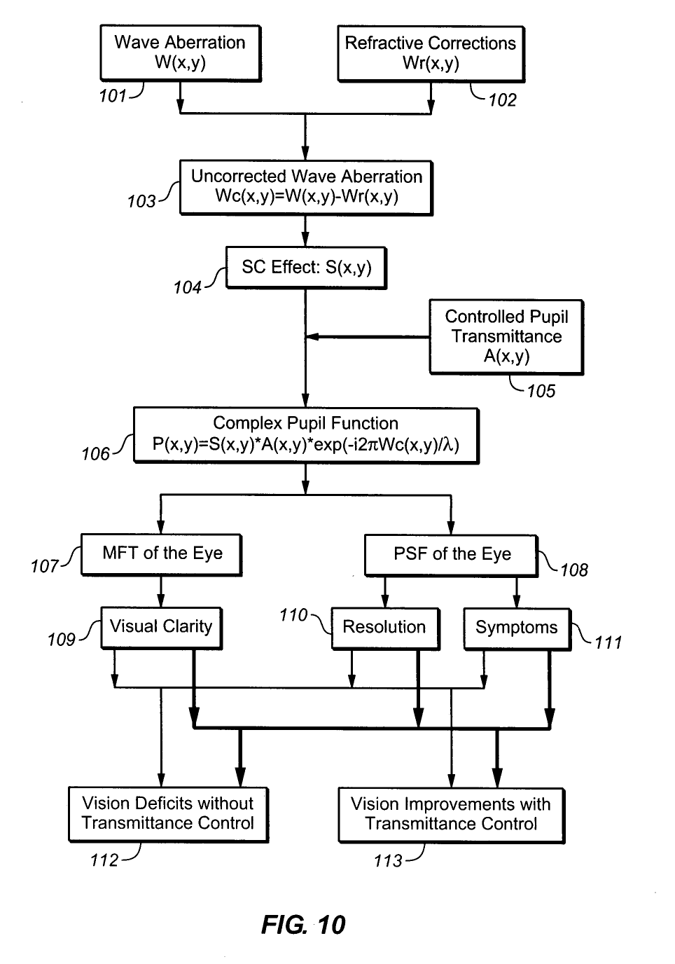

FIG. 10 shows a process for selecting or prescribing a lens with wavefront-optimized pupil transmittance in accordance with one embodiment of the invention. First, the wave aberration of an eye 101 is measured with a wavefront aberrometers such as a Hartmann-Shack wavefront sensor for the eye. Second, residual (uncorrected) wave aberration of eye 103 is determined by removing a refractive correction 102, wherein the refractive correction may include defocus and astigmatism in the eye known in the prior art. The uncorrected wave aberration is calculated according to the following formula.
Wc(x,y)=W(x,y)−Wr(x,y) where

Wc is a 2D distribution of uncorrected wavefront error of an eye,

x is the Cartesian x-coordinate across pupil of the eye

y is the Cartesian y-coordinate across pupil of the eye

W is the wavefront error that includes all aberrations in an eye

Wr is the corrected wavefront errors such as focus error and astigmatism. These descriptions apply throughout all equations described herein.

Third, a complex pupil function across pupil of the eye 106 is obtained by combining the uncorrected wave aberration 103 and an amplitude transmittance function, including the Stiles-Crawford (SC) effect 104 and a pupil transmittance function 105 for a lens with CPT. The complex pupil function is determined according to the following equation.
P(x,y)=S(x,y)*A(x,y)*exp(i2πWc(x,y)/λ) where

P is the complex pupil function across pupil of the eye

x is the Cartesian x-coordinate across pupil of the eye

y is the Cartesian y-coordinate across pupil of the eye

* is a multiplication operator

S is the amplitude transmittance according to Stiles-Crawford effect

A is an amplitude transmittance across pupil of the eye

Exp is a exponential function

i represents a complex operator

Wc is a 2-Dimensional distribution of residual wavefront error of an eye

λ is the wavelength of visible light.

These descriptions apply throughout all equations described herein.

For scotopic vision, Stiles-Crawford effect can be ignored and S(x,y)=1. Fourth, parameters for retinal image quality of the eye can be derived from the complex pupil function according diffraction theory, including the Modulation-Transfer Function (MFT) of the eye 107 and the Point-Spread Function (PSF) of the eye 108. Fifth, vision of eye with CPT and without CPT is evaluated. Metrics for vision evaluation may include Vision Clarity (VC) 109 as a relative MTF score within a population as disclosed in U.S. patent application Ser. No. 11/370,745, entitled Methods for Specifying Quality of Vision from Wavefront Measurements, filed on Mar. 8, 2006 by J. Liang, and which published on Oct. 19, 2006 as U.S. Patent Publication No. 2006/0232744, Aberration-Induced Symptoms (AIS) 111 as disclosed in U.S. patent application Ser. No. 11/371,288, entitled Algorithms and Methods for Determining Aberration-Induced Vision Symptoms in the Eye from Wave Aberration, filed on Mar. 8, 2006 by J. Liang, and which published on Sep. 14, 2006 as U.S. Patent Publication No. 2006/0203198, and visual resolution 110. Visual resolution can be estimated by convolving an acuity chart with the eye's point-spread function, and assessing the convolved images based on legibility and retinal contrast. Sixth, vision deficits of conventional lens are identified 112 and improvements in vision by a lens with wavefront-optimized pupil transmittance are specified 113. Seventh, a wavefront-optimized pupil transmittance is identified for a lens with controlled profile transmittance (CPT). Eighth, a prescription for a lens with wavefront-optimized pupil transmittance is obtained, comprising a wavefront-optimized pupil transmittance and a refractive correction. In order to address the issue of accommodation in a wavefront measurement, the refractive correction of the lens can be modified according to a manifest refraction. Ninth, the prescription of a lens with CPT is transmitted to a lens making system. Lenses with wavefront-optimized pupil transmittance can be made for contact lenses, Phakic intro-ocular lenses and intro-ocular lenses. Finally, vision of an eye using a lens with wavefront-optimized pupil transmittance is specified for a broad metrics such as Vision Clarity, and visual resolution (acuity), and aberration-induced symptoms. The disclosures of U.S. Patent Publication No. 2006/0232744 and U.S. Patent Publication No. 2006/0203198, which are described above, are hereby incorporated herein by reference in their entirety.

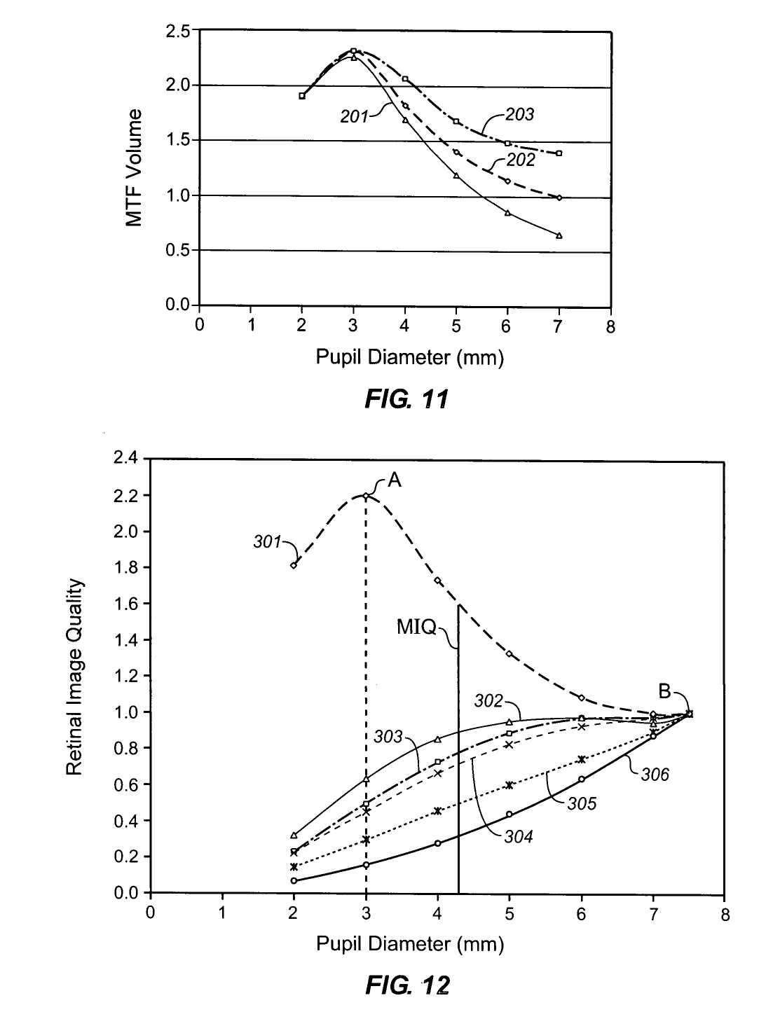

Controlling pupil transmittance can be achieved by two approaches: by controlling pupil size of the eye or by altering energy transmittance across the pupil. Stiles-Crawford effect is an example that changes transmittance across the pupil for photopic vision. Simulations were conducted to determine the efficiency in improving vision by controlling pupil size and by altering pupil transmittance according to the standard Stiles-Crawford effect. For same luminance efficiency across the pupil, controlling pupil size is found twice as efficient as altering the transmittance with Stiles-Crawford effect as shown in FIG. 11. Image quality of the eye is measured by the total volume under the MTF of the eye up to 60 c/deg. The averaged image quality for 21 eyes with known wave aberrations is shown for a uniform pupil transmittance 201, for a pupil transmittance according to the standard Stiles-Crawford effect 202, and for controlled pupil size that has the same luminance efficiency as the Stiles-Crawford effect 203.

Controlling pupil size is thus the preferred embodiment for lenses with controlled pupil transmittance. Another advantage of lenses with controlled pupil size is the possibility to combine pupil size control with cosmetic control of iris color for contact lenses and Phakic intraocular lenses. Lenses can be made to improve vision at night and to change iris color during the day.

Methods for Specifying Retinal Intensity and Retinal Brightness of Objects for Lenses with Controlled Pupil Transmittance (CPT)

True performance of a lens with CPT is not completely specified without knowing impacts of reduced pupil transmittance on retinal brightness and retinal intensity. Retinal intensity represents brightness of a point source like a star. It depends on the distance between the source and the observers. Retinal brightness represents brightness of an extended object like the moon. Retinal brightness is independent of the distance from the object to the observers if the extended object is far bigger than the scale of point-spread functions of the eye.

Referring to FIG. 12, retinal image quality, retinal intensity and retinal brightness of averaged normal human eyes for a plurality of pupil sizes is graphically shown. Retinal image quality of the eye measured by eye's MTF volume with Stiles-Crawford effect (photopic vision, i.e., cone vision) is shown by the curve 301. Retinal intensity of a point source is shown by the curve 302 for cone vision with Stiles-Crawford effect, and by the curve 303 for rod vision without Stiles-Crawford effect. Retinal brightness of an object is shown by the curve 304 for cone vision with Stiles-Crawford Effect and the curve 305 for rod vision without Stiles-Crawford effect. Retinal brightness of a large extended object without Stiles-Crawford effect for scotopic vision is shown by the curve 306. Data are derived from measurements of wave aberrations, averaged for 21 healthy normal eyes, and normalized to the values at the largest natural pupil (B). In the calculation, focus error and cylindrical error in the 21 eyes for each pupil size is calculated and removed from the wave aberration of the eyes.

On average, optical quality of the eye is low at a small pupil and increases as pupil size increases up to about 3 mm, and decreases as the pupil size gets bigger. Pupil size with the best image quality is different from eye to eye between 1.5 mm and 4 mm. Average retinal intensity and retinal brightness increases as pupil size of the eye increases as expected. However, important findings about retinal intensity (brightness) must be notices as follows.

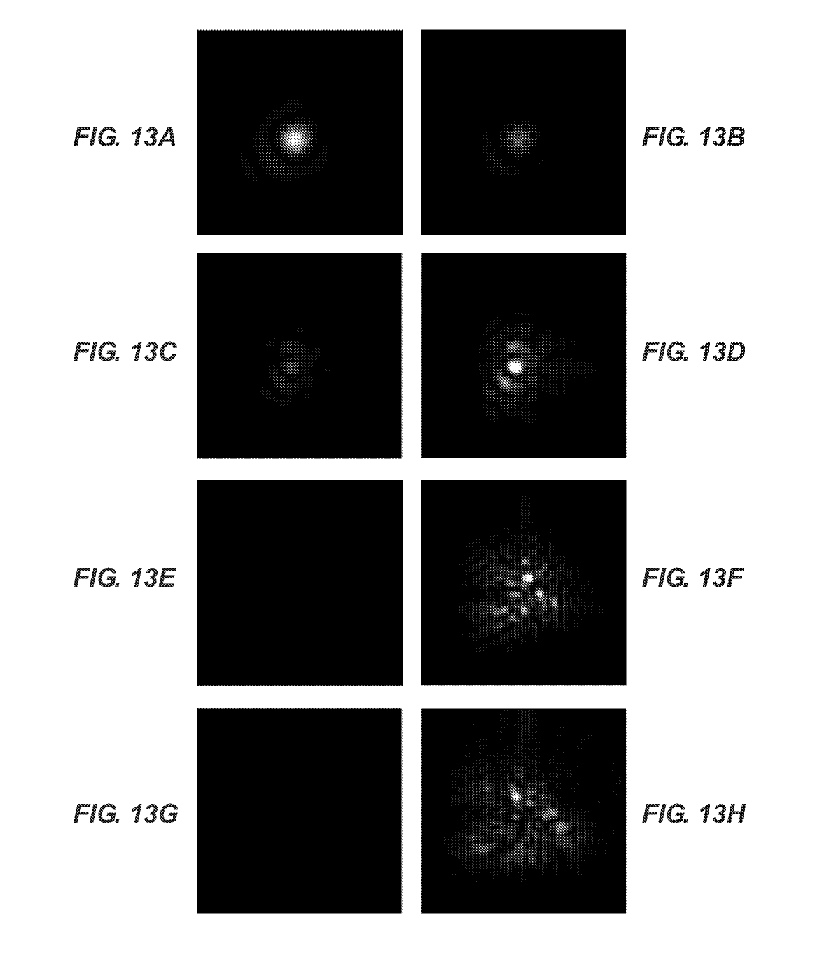

First, retinal intensity of a point source increases significantly from 2 mm to about 4 mm (as much as 300%) as expected whereas the change in retinal intensity above 4 mm is not significant, which is not known before. On average for 21 eyes, the change in retinal intensity above 4 mm is only about 20% for photopic (cone) vision and about 30% for scoptopic (rod) vision. The primary reason for insignificant change in retinal intensity above a 4 mm pupil is the image blur caused by uncorrected high-order aberration in the eye. This can be demonstrated by displaying point-spread functions of a real eye in FIG. 13A-H. Referring to FIGS. 13A,C,E, and G, the retinal point-spread functions of an eye from the 21 eyes in our study for a pupil diameter of 2 mm (FIG. 13A), 4 mm (FIG. 13C), 6 mm (FIG. 13E) and 7.7 mm (FIG. 13G). These point-spread functions are normalized to the peak intensity of an aberration-free eye at the same pupil size. The peak intensity is the so-called Strehl Ratio. The Strehl ratio for an eye decreases as pupil size increase because of increased image blur caused by the high-order aberrations in the eye. Referring to FIGS. 13B, 13D, 13F, and 13H the retinal point-spread images of the same eye is shown for a pupil diameter of 2 mm (FIG. 13B), 4 mm (FIG. 13D), 6 mm (FIG. 13F) and 7.7 mm (FIG. 13H), but scaled according to the total energy for each pupil dimension. Total energy for a point source increases as pupil size increases. However, retinal intensity does not increase so much above 4 mm because high-order aberrations in the eye causes significant image blur over a large retinal area. In many cases, the peak retinal intensity decreases as pupil size is larger than a certain pupil dimension. One should not use the peak intensity as the measure of retinal intensity because human retinal has a property of retinal summation over a certain retinal area. Accordingly, the intensity of a point object shown in FIG. 12 is not peak intensity of the retinal point image, rather is determined by integrating the energy in an area around the peak in the PSF.

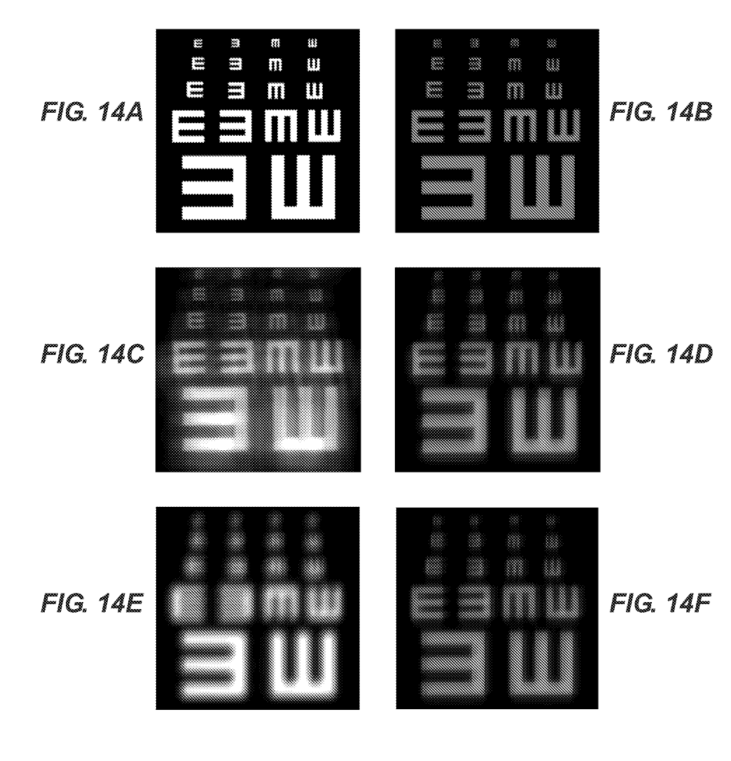

Second, retinal brightness for photopic (cone) vision increases as much as 250% from 2 mm to 4 mm, but it chances only about 30% above 4 mm because of Stiles-Crawford effect and image blur caused by high-order aberrations. It must be emphasized that brightness also depends on object size if the extended object is not much larger than the point-spread function. FIG. 14A and FIG. 14B show retinal images of an acuity chart of an aberration-free eye for a pupil size of 6 mm and for a pupil size of 3 mm, respectively. As expected, when the eye is aberration-free, retinal brightness of the acuity letter is same for all letter sizes, and lower at a small 3 mm pupil. Retinal images for two real eyes of different persons are also shown for a pupil size of 6.7 mm pupil (FIG. 14C, FIG. 14E) and for a 3.75 mm pupil (FIG. 14D, FIG. 14F). Brightness in real eyes at a large 6.7 mm pupil is higher for the large letters and lower for the smaller letters in FIG. 14C and FIG. 14E due to the image blur caused by high-order aberrations in the eye. Retinal brightness for a small object behaves more like intensity of a point source. Additionally, brightness of acuity letters is about the same for a pupil size around 3.75 mm for the ideal eye as well as for the real eyes. When pupil size is reduced from a large 6.7 mm pupil to a medium 3.75 mm, reduced retinal brightness is noticeable for the larger letters (20/80 for the largest letters), but insignificant for the smaller letters.

Third, change in retinal brightness for scotopic vision is significant from 2 mm to largest natural pupil. Retinal brightness is proportional to pupil area for a large extended object 306 and is proportional to pupil diameter. Performance for scotopic (rod) vision was of critical importance for human when lived in caves, but is of much less importance for modern life with man-made electric light sources everywhere. Generally speaking, we could ignore retinal brightness for scoptopic vision for design of refractive vision corrections because scotopic vision matters in real life only when people sleep with the lights off.

We have shown the characterization of retinal brightness and retinal intensity for an averaged eye along with retinal image quality that is measured by an eye's MTF. Retinal brightness and retinal intensity for a real eye can be derived from wave aberration in the eye and a known pupil transmittance function for a refractive correction, comprising: obtaining wave aberration of an eye at a large natural pupil with a wavefront aberrometers like a Hartman-Shack sensor for the eye; determining an uncorrected aberration in the eye by removing a refractive correction from the obtained wave aberration, wherein the refractive correction may include defocus and astigmatism in the eye; calculating a complex pupil function based on the determined uncorrected aberrations in the eye and a pupil transmittance profile across the pupil of the lens and Stiles-Crawford effect if photopic vision is concerned; calculating the retinal point-spread function from the complex pupil function of the eye; determining a relative retinal intensity and brightness. For the determination of retinal intensity, an integrated energy around the peak of the point-spread function (encircle energy) can be specified to simulate retinal summation. For the determination of retinal brightness, brightness of an object can be determined from integrated energy around the peak of a line-spread function (culminated energy), determined from the calculated point-spread function. Alternatively, retinal image of acuity chart can be calculated and displayed by convolving the point-spread function of the eye with an acuity chart. Brightness for each acuity letter can be estimated from the convolved retinal image.

Determination of retinal intensity and retinal brightness can be used for clinical evaluations of lenses with controlled pupil transmittance. It can also be used to guide designs of lenses with controlled pupil transmittance.

Methods for Wavefront-Optimized Lens (WOL) with Controlled Pupil Sizes

Once obtaining the retinal image quality and the retinal intensity (brightness) together in FIG. 12, one can make an intelligent design for a wavefront-optimized lens by optimizing vision between retinal image quality and retinal intensity.

Performance of human eyes at their natural pupil size can be argued to be best suited for human life in the cave age. For scotopic (rod) vision, human eye achieves high photon efficiency through a large pupil size (8 mm) and with rod as photoreceptors. For photopic vision, human eye achieves the best image quality for a small pupil around 3 mm and the Stiles-Crawford effect for cone vision improves retinal image quality at a large pupil without impact on scotopic retinal brightness.

With the introduction of man-made electric light sources, performance for scotopic vision can be sacrificed to a great extent for improved photopic vision because photopic vision with a large natural pupil at night suffers from night vision symptoms such as glare, halo, ghost images and starburst. If loss in retinal intensity and retinal brightness for photopic vision is not significant, reducing effective pupil size of the eye can improve night vision significantly.

If vision clarity and visual acuity were the only determining factor for a refractive correction, vision optimization should be targeted for the Best Optical Quality (BOQ) in all possible pupil sizes. However, the BOQ optimization could be problematic because many normal human eyes have best optical quality for a pupil size less than 2.5 mm. First, significant loss in retinal brightness and retinal intensity are expected for many eyes. Loss in retinal brightness and retinal intensity can be as much as 300% for a pupil size of 2.5 mm. Second, contact lenses with a pupil size of 2.5 mm can reduce field of view for day vision.

In a more balanced approach, one would take in account of image quality, retinal intensity, and retinal brightness for photopic vision, and select an optimized pupil size smaller than the largest natural pupil and larger than the pupil size with the best optical quality. A number of approaches can be devised based on this principle. In one embodiment, the optimized pupil size can be the average of the largest natural pupil and the pupil size with the best optical quality. In another embodiment, one can find a pupil size that gives the highest product of retinal image quality (MTF volume) and retinal intensity. One preferred embodiment is to find an optimized pupil size that leads to the Median Image Quality (MIQ), being the average of the optical quality at the largest natural pupil “A” and the best image quality for the eye “B.” The MIQ optimization leads to about 60% improvement in retinal image quality along with a loss in retinal intensity less than 20% and in photopic brightness of about 30%, according to the results in FIG. 12.

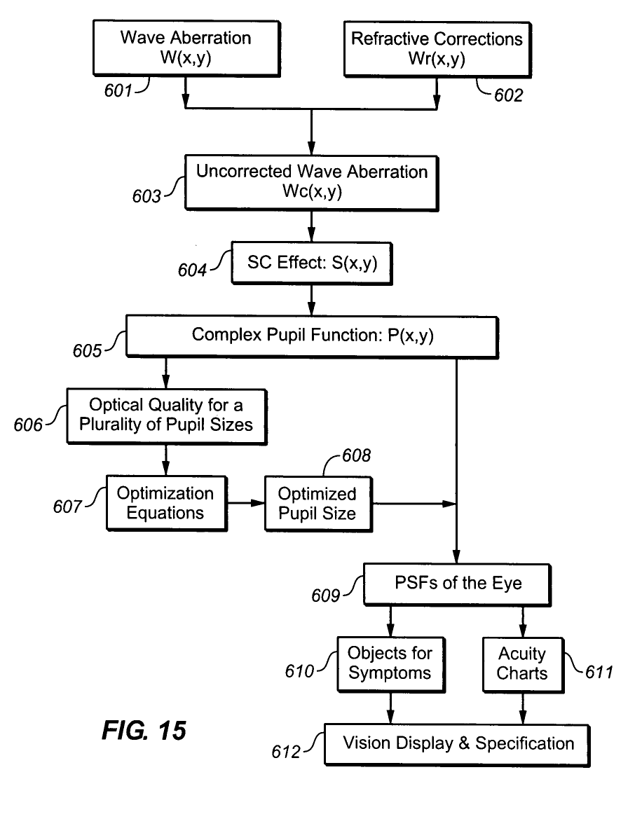

Referring to FIG. 15, an example of implementation of the MIQ optimization is shown. In this example, the implementation comprises obtaining a wave aberration of an eye at a large natural pupil 601; determining a conventional refractive correction 602 and uncorrected aberrations in the eye 603; calculating a complex pupil function 605 from the uncorrected wave aberration 603 and Stiles-Crawford effect; determining a retinal image quality of the eye for a plurality of pupil sizes 606, wherein the retinal image quality can be the total volume under modulation transfer function of an eye and the refractive corrections for each pupil size can be different in the calculation; finding the values of the best image quality (the largest MTF volume) and the image quality (MTF volume) for the natural pupil and calculating the median image quality as the average of the highest and lowest image quality 607; finding a pupil size that has a retinal image quality (MTF volume) matching to the calculated median image quality 608, wherein the found pupil size is smaller than the natural pupil size of the eye and larger than the pupil size with the best optical quality.

Although the MIQ optimization can provide excellent and balanced performance for night vision for most eyes, other aspects should also be considered. Vision optimization according to the MIQ optimization may not eliminate night symptoms completely for some eyes. For therapeutic treatments, typically it is important that improved vision clarity can solve problems for night vision such as night symptoms and low acuity at night.

Night vision of eyes using a lens with controlled pupil size and using a conventional lens with a natural pupil can be studied and displayed based on the complex pupil functions for an optimized pupil 608 and for the largest natural pupil 605. Retinal point-spread functions of the eye can be derived for a conventional lens and for a lens with an optimized pupil size 609. Aberration-Induced Symptoms 610 can be derived by convolving the point-spread functions of the eye with objects specially designed for aberration-induced symptoms such as glare, halo, and ghost images as disclosed in U.S. patent application Ser. No. 11/371,288, entitled Algorithms and Methods for Determining Aberration-Induced Vision Symptoms in the Eye from Wave Aberration, filed on Mar. 8, 2006 by J. Liang and cited above. Acuity at night can also be estimated by convolving the point-spread functions of the eye with a night acuity chart (bright letter on black background) 611.

Night vision of an eye can be further optimized based on the results of aberration induced symptoms 610 and the calculated night acuity 611 by choosing a new optimized pupil that is larger or smaller than that obtained from the MIQ optimization. Night symptoms can be completely eliminated by a wavefront-optimized lens for therapeutic treatments if the optimized pupil size is small enough.

Lenses with Wavefront-Optimized Pupil

Night vision for 21 eyes with known wave aberration was studied for a conventional lens with a large natural pupil and for a lens with a wavefront-optimized pupil according to MIQ optimization. Night vision of most eyes in the study are improved with a wavefront-optimized pupil. Improvements in night vision can be qualified as improved night acuity, and/or reduced or eliminated aberration-induced symptoms if symptoms are at present in the large natural pupil.

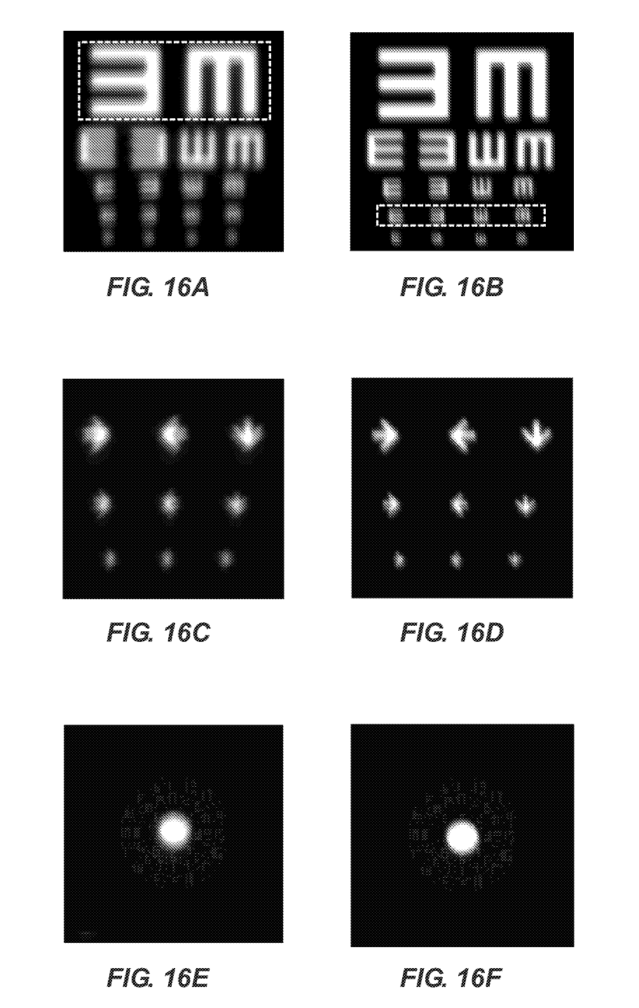

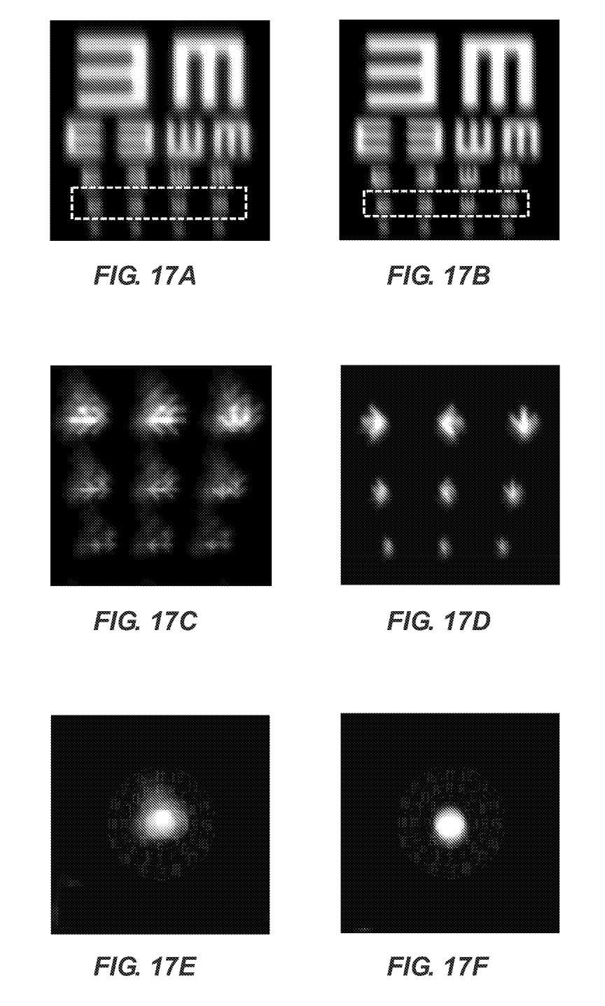

As an illustration, FIGS. 16A-F and FIGS. 17A-F show night vision for 2 eyes in this study. The sizes of the natural pupil for these two eyes are respectively 6.7 mm and 7.24 mm, while the optimized pupil sizes are respectively 3.75 mm and 5 mm according to the MIQ optimization. The images in FIGS. 16A,C, and E and FIG. 17A,C, and E are for vision with the large natural pupil while images on the right are for vision with the optimized pupil for the same eye. For the patient shown in FIGS. 16A-F, night resolution is improved from 20/80 (largest letters) for the large natural pupil of 6.7 mm in FIG. 16A to 20/15 (second smallest letters) for a MIQ pupil of 3.75 mm in FIG. 16B. Halo in FIG. 16C and FIG. 16E for the large natural pupil is eliminated as shown in FIG. 16D and FIG. 16F for the optimized pupil. Improvement in night vision for the eye shown in FIGS. 16A-F should increase response time significantly for night driving.

For the patient shown in FIGS. 17A-F, improved vision clarity leads to elimination of ghost images in FIG. 17C and significant night glare in FIG. 17E. Night life as well as night driving for the eye in FIG. 8 would be unacceptable with the large natural pupil. But with a wavefront-optimized pupil of 5 mm, aberration-induced symptoms of ghost image and halo can be eliminated completely.

Table 1, which follows, shows the gains in vision clarity at night, defined as ratio of MTF with wavefront-optimized pupil to MTF volume with natural pupil, as well as the natural pupil size and the wavefront optimized pupil according to MIQ optimization for all 21 eyes in this study. On average, gain in MTF volume is about 100% instead of 60% shown in FIG. 12 because FIG. 12 uses the averaged MTF volume of 21 eyes for the same pupil size.

It is important to notice that the wavefront optimized pupil size according to MIQ optimization has a narrow distribution with a mean of 4.3 mm and a standard deviation of only 0.6 mm. In one preferred embodiment, a lens for Wavefront-Optimized Pupil (WOP) can be fabricated, comprising: a transparent inner optical zone for a standard refractive correction of defocus and astigmatism; an opaque outer segment that limits the effective pupil size of an eye at night to about 4.3 mm, or larger than 3.6 mm and smaller than 5 mm. Such WOP lenses will improve night vision significantly for the normal population without noticeable loss in retinal intensity and brightness, as shown in FIG. 12.

TABLE 1
Table Gain in Vision Clarity for Night
Vision by Wavefront Optimized Pupil

| Patient | Gain in Vision | Natural Pupil | MIQ Pupil | 85% Natural |
|---|---|---|---|---|
| ID | Clarity | Size (mm) | Size (mm) | Pupil (mm) |
| 1 | 1.51 | 7.00 | 3.25 | 5.95 |
| 2 | 1.74 | 7.24 | 4.75 | 6.15 |
| 3 | 1.84 | 6.43 | 3.50 | 5.47 |
| 4 | 1.75 | 6.70 | 3.75 | 5.70 |
| 5 | 3.40 | 7.00 | 4.50 | 5.95 |
| 6 | 1.63 | 7.00 | 4.00 | 5.95 |
| 7 | 2.45 | 7.80 | 4.00 | 6.63 |
| 8 | 1.38 | 6.70 | 5.00 | 5.70 |
| 9 | 1.86 | 7.24 | 5.00 | 6.15 |
| 10 | 2.11 | 6.70 | 3.75 | 5.70 |
| 11 | 1.76 | 7.24 | 4.25 | 6.15 |
| 12 | 2.07 | 6.70 | 4.00 | 5.70 |
| 13 | 2.49 | 6.70 | 3.75 | 5.70 |
| 14 | 1.36 | 7.50 | 5.25 | 6.38 |
| 15 | 2.89 | 6.70 | 4.25 | 5.70 |
| 16 | 1.68 | 7.80 | 5.00 | 6.63 |
| 17 | 2.08 | 6.70 | 5.50 | 5.70 |
| 18 | 2.00 | 6.43 | 4.25 | 5.47 |
| 19 | 1.95 | 7.50 | 3.75 | 6.38 |
| 20 | 1.74 | 7.00 | 4.24 | 5.95 |
| 21 | 3.19 | 7.80 | 4.75 | 6.63 |
| Max | 3.40 | 7.80 | 5.50 | 6.63 |
| Min | 1.36 | 6.43 | 3.25 | 5.47 |
| Mean | 2.04 | 7.04 | 4.31 | 5.99 |
| Sigma | 0.56 | 0.44 | 0.61 | 0.37 |

According to the foregoing example, one ophthalmic device embodiment according to the invention comprises a contact lens as described above with a central portion, e.g., central portion 413 (FIG. 4C) having an outer diameter of 3.25 mm to 5.50 mmn. In the alternative, the outer annular portion, e,g, portion 412 (FIG. 4C) has an inner diameter of 3.25 to 5.50 mm.

In another embodiment, lenses for Wavefront-Optimized Pupil (WOP) are fabricated, comprising: a transparent inner optics for a standard refractive correction of defocus and astigmatism; an opaque outer segment that limits effective pupil size to a plurality of dimensions such as 3.75 mm, 4 mm, 4.25 mm, 4.5 mm, 4.75 mm and 5 mm. The lenses with WOP are further sold to optometrists or clinical practitioners for clinical evaluation of human vision or sold to individual consumers based on a customized clinical prescription containing not only refractive corrections but also dimensions of the central optics.

In yet another embodiment, a lens or lenses with Wavefront-Optimized Pupil (WOP) are custom made comprising: a transparent inner optics for a standard refractive correction of defocus and astigmatism; an opaque outer segment that limits effective pupil size of the eye at night, wherein the dimension of the opaque segment is custom determined based on wave aberration of individual eyes. The night vision optimization comprises obtaining a wave aberration of an eye, calculating retinal image performance at night for a plurality of pupil sizes, and selecting an optimized pupil size based on any one or combination of night acuity, vision clarity at night, and aberration-induced symptoms.

All embodiments of lenses with Wavefront-Optimized Pupil (WOP) in the present invention are suited for contact lens and lens implemented in the eye. Additionally, wavefront-optimized contact lenses and Phakic introcular lenses can be combined with cosmetic control of iris color.

Methods and Systems for Estimating Visual Acuity from a Wavefront Measurement

Having an ability to predict visual acuity of eye is critically important for vision design and vision diagnosis. From a wave aberration of an eye, it is easy to derive a retinal image of any optical object. It is well-known in diffraction theory that point-spread function of an optical system can be derived from a wave aberration and image of an optical object is the convolution of the point-spread function and a selected optical object. Simulating image properties of an image element (system) is a standard function in most commercial software used in optical design, wherein the simulated images are obtained by calculating a point-spread function of the designed element, convolving a letter with the calculated point-spread function, and displaying the convolved image for visual inspection. A human eye is an optical system and can be constructed in lens design software and performance of an eye can be the can be simulated based on wave aberration of an eye. Calculating a retinal image of a real eye from an objective measurement of wave aberration was first published by Artal in “Calculations of two dimensional fovea retinal image in real eye,” Journal of Optical Society of America A, vol. 7, pp 1374-1381, 1990. The process comprises obtaining an objective measurement of wave aberrations of a living human eye; calculating an intensity point-spread function of eye from the wave aberration; obtaining the retinal image of an optical object by convolving the calculated point-spread function with the optical object. Since vision of eye is often evaluated with an acuity chart, it would be obvious to everyone having ordinary skill in the art to derive the retinal image of an acuity chart by convolving an acuity chart with a calculated point-spread function.

Although retinal images of acuity charts can be easily derived from wave aberration of the eye, estimation of visual acuity remains to be a challenging task. Technologies in the prior art did address two fundamental issues for acuity estimation. First, retinal image of an eye in acuity measurement is not known because an eye's wave aberration at the exact pupil size at which visual acuity is measured is not known. Wave aberration of an eye is often measured at a very low light level for a pupil size as large as possible. Thus, the pupil size of an eye at wavefront measurement can be significantly different from that of eye in clinical acuity tests. Second, image processing by the retina and the brain is too complicated to be simulated automatically.

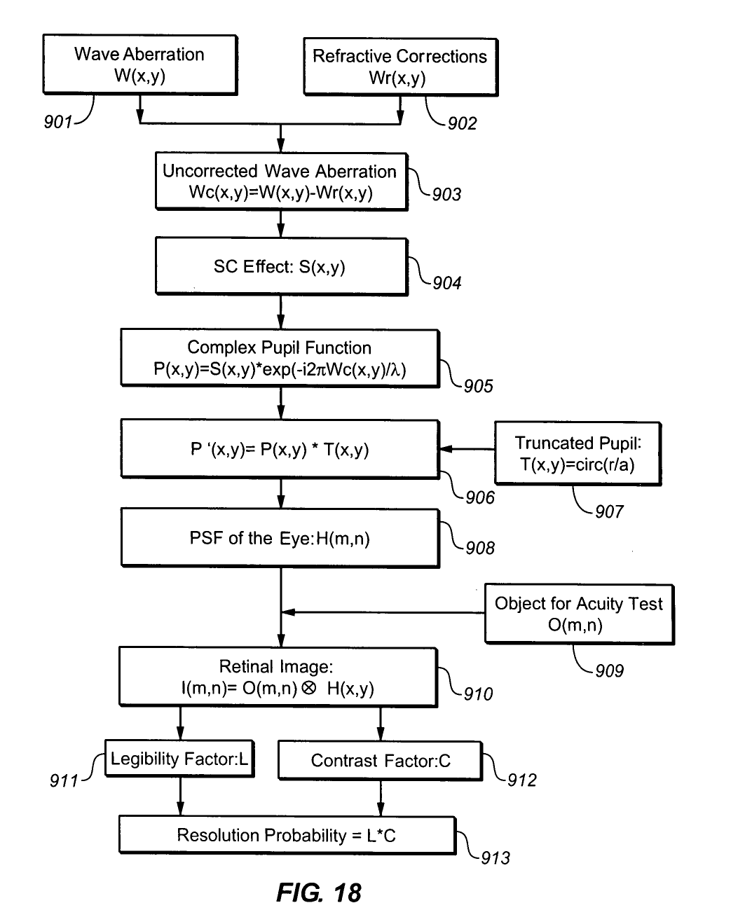

Referring to FIG. 18, a block diagram illustrating a procedure for estimating visual acuity of an eye from wave aberration of an eye according to one embodiment of the invention is shown. The procedure steps are: (1) Obtain a wave aberration of an eye at the condition of acuity measurement is obtained by factoring in the difference between the pupil size at the wavefront measurement and the pupil size in measuring visual acuity. A measured pupil size of an eye during an acuity measurement can be used to scale-down the pupil size of a wavefront map obtained from a wavefront measurement of the eye at low light level. Alternatively, a fixed scale-down factor can be applied to the pupil size of the measured wavefront as shown in the last column of Table 1, a fixed reduction of 15% of pupil size from the measured natural pupil size for 21 eyes. The reducing factor should be practically determined based on the luminance level in acuity measurements and in wavefront measurements. (2) Image processing by the retina and the brain is represented by a contrast factor and a legibility factor. The contrast factor can be determined from the retinal image and a pre-determined retinal contrast threshold. If the contrast of the retinal image is above a required contrast threshold, the contrast factor is 1. Otherwise, the contrast factor is zero. The legibility factor is subjectively determined by presenting the calculated image of an acuity chart to a human observer. If the acuity letter can be read correctly, the legibility factor is 1. Otherwise, the legibility factor is zero. The legibility factor in this process will be affected by reduced image contrast as well as distortion of letters by the image blur in the eye. The product of the legibility factor and the contrast factor determines whether an acuity letter is recognizable or not.

Returning to FIG. 18, one method according to the invention comprises: obtaining a wave aberration of an eye 901 from a wavefront aberrometer like a Hartmann-Shack wavefront sensor for the eye, wherein the wave aberration has a scale-down factor for the effective pupil size; determining a residual (uncorrected) wave aberration of eye 903 by removing a refractive correction 902, wherein the refractive correction may contain defocus and astigmatism; deriving a complex pupil function across pupil of the eye 905 [P(x,y)=S(x,y)*exp(i2πWc(x,y)/λ)] by combining the uncorrected wave aberration 903 and the Stiles-Crawford effect 904; calculating a new complex function 906 [P′(x,y)=P(x,y)*T(x,y), where P′ is a truncated complex pupil function for the controlled pupil transmittance, P is the complex pupil function of the eye, T is a circular function representing the controlled pupil size] if a lens with controlled pupil size is used for a vision correction by combining 905 with a pupil truncation function 907 [T(x,y)=circ(r/a), where circ is a circular function, r is the radius within eye's pupil, and a is the radius of the optimized pupil] with a radius a; calculating a point-Spread Function (PSF) of the eye 908 [H(m,n), where m is the pixel number in x-axis and n is the pixel number in y-axis]; calculating a retinal image 910 [I(m,n)=O(m,n) H(x,y) where I is the convoluted image, being the convolution of O(m,n) and H(x,y)] by convolving the calculated point-spread function 908 with an object for acuity test 909 [(O(m,n), where O is the optical object like an acuity chart]; determining the legibility factor “L” 911 and a contrast factor “C” 912 from the calculated retinal image 910; estimating visual acuity of the eye based on acuity probability, being the product of the legibility factor and the contrast factor.

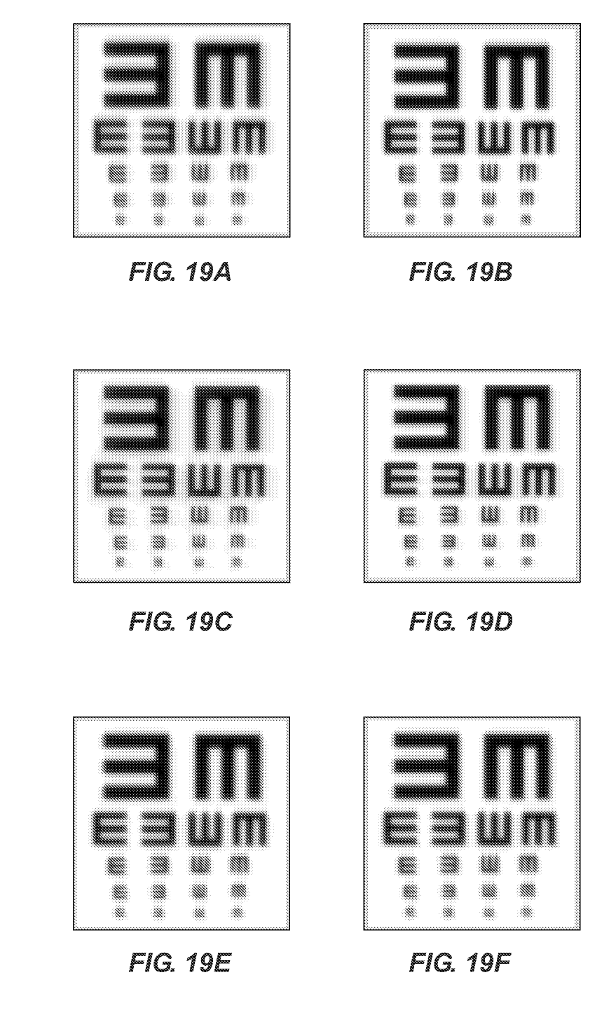

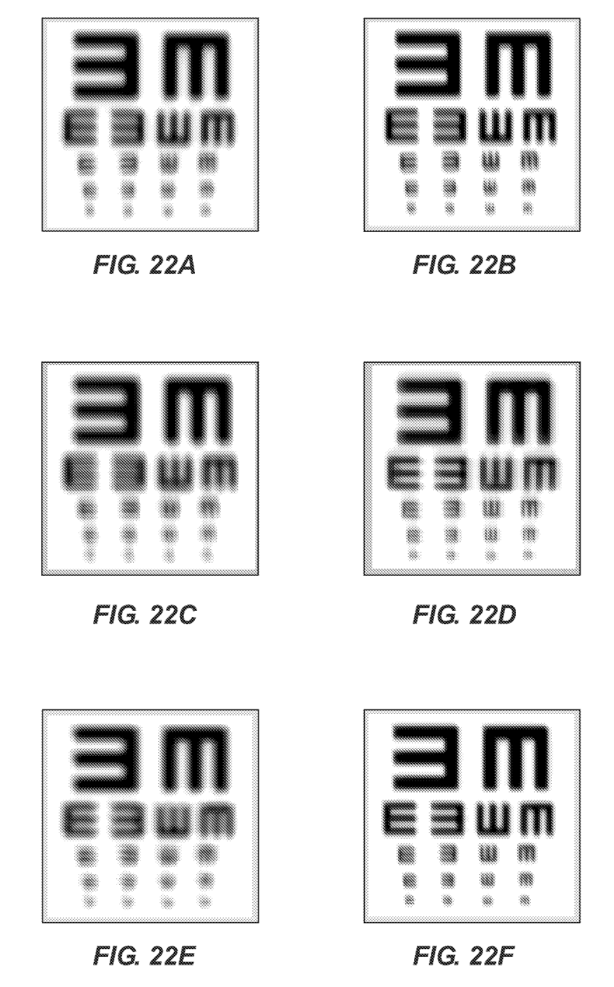

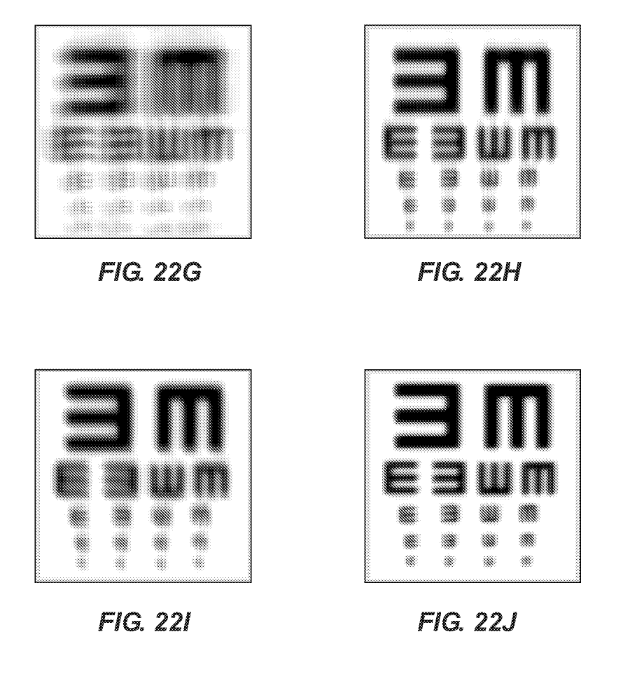

The method illustrated in FIG. 18 for estimating visual acuity of an eye is applied to the 21 eyes listed in Table 1 for an investigation of potential improvements in acuity by the MIQ optimization. For eyes with a conventional vision correction, pupil sizes of the 21 eyes are listed in the last column in Table 1. Instead of using the natural pupil (column 3) measured by the wavefront sensor, 85% of the natural pupil (last column) is used for acuity estimation in order to factor in the difference between the luminance levels in the wavefront measurements and in an acuity test. For the eyes using a lens with a wavefront-optimized pupil, the pupil size of the eye is listed in column 3 in table 1, determined according to the MIQ optimization. For simplicity, we estimate visual acuity based on the legibility factor only, because the contrast factor is by design better with a wavefront-optimized pupil. Retinal images of 21 eyes with a conventional lens and with a wavefront-optimized lens are shown in FIGS. 19A-F through FIGS. 22A-J. One can easily conclude that vision of 21 eyes with the conventional refraction is different from eye to eye whereas vision of the same 21 eyes with a wavefront-optimized pupil is consistent and better than that with conventional lenses for most eyes. Under a correction with a wavefront-optimized pupil, most eyes can have an acuity of 20/15 (second smallest letters) and about 90% (19 out 21) of eyes can have an acuity of 20/10. This is a remarkable improvement compared to vision with a conventional sphero-cylindrical correction. Four different categories of eyes are found for the 21 eyes in this study.

First, negligible difference was found for 3 out of 21 eyes (about 14%). Retinal images on the left (FIG. 19A, 19C, 19E) are for the eyes with a conventional correction while retinal images on the right (FIG. 19B, 19D, 19F) are for the same eyes with a lens with wavefront-optimized pupil according to the MIQ optimization.

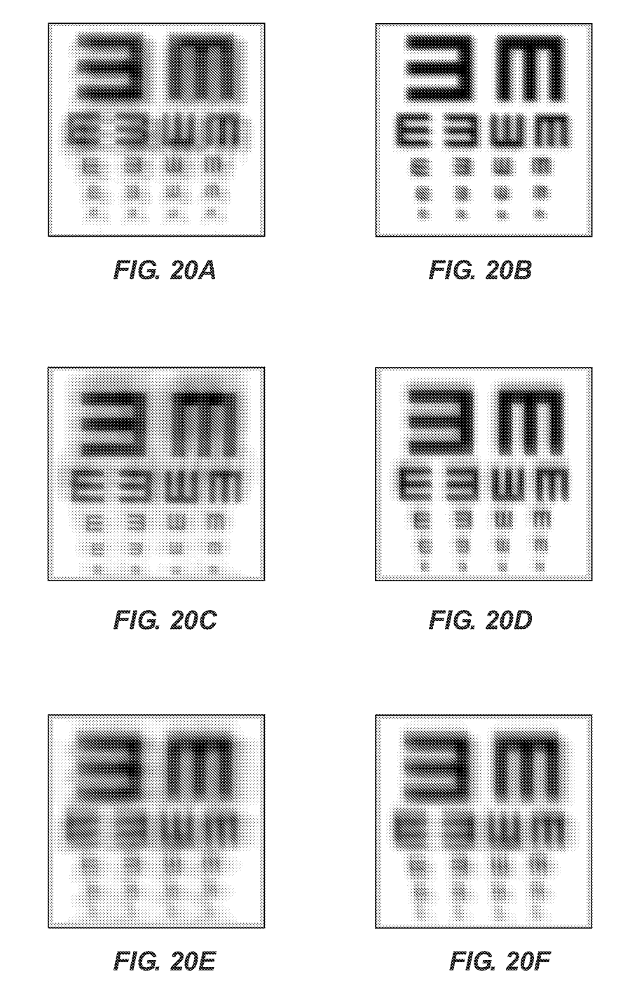

Second, improved vision clarity without changing in acuity was found for 3 out of 21 eyes (about 14%). Retinal images on the left (FIG. 20A, 20C, 20E) are for the eyes with a conventional correction while retinal images on the right (FIG. 20B, 20D, 20F) are for the corresponding eyes using a lens with wavefront-optimized pupil according to the MIQ optimization.

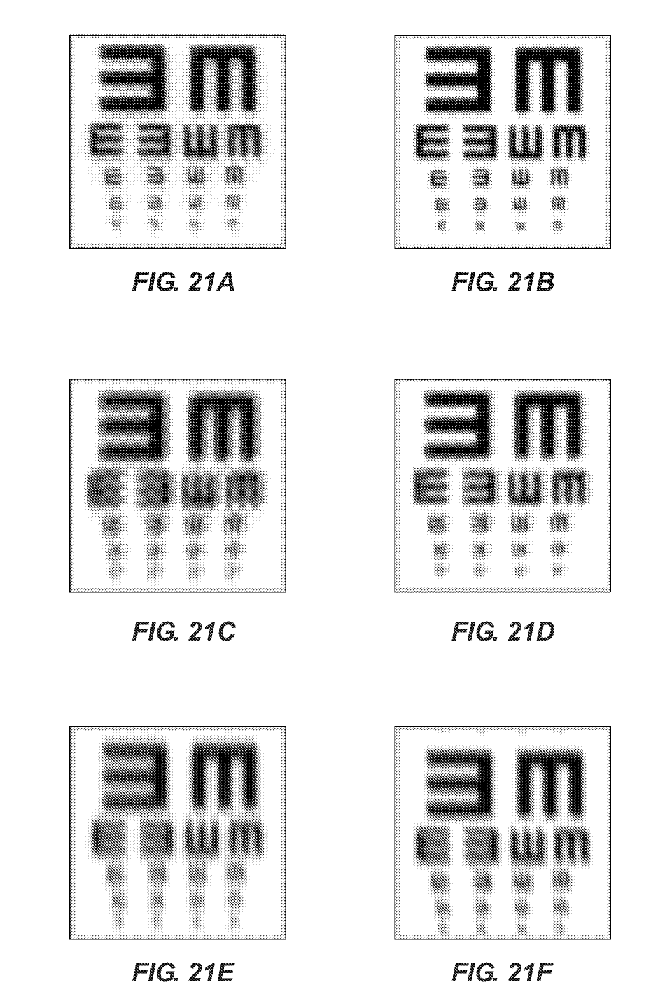

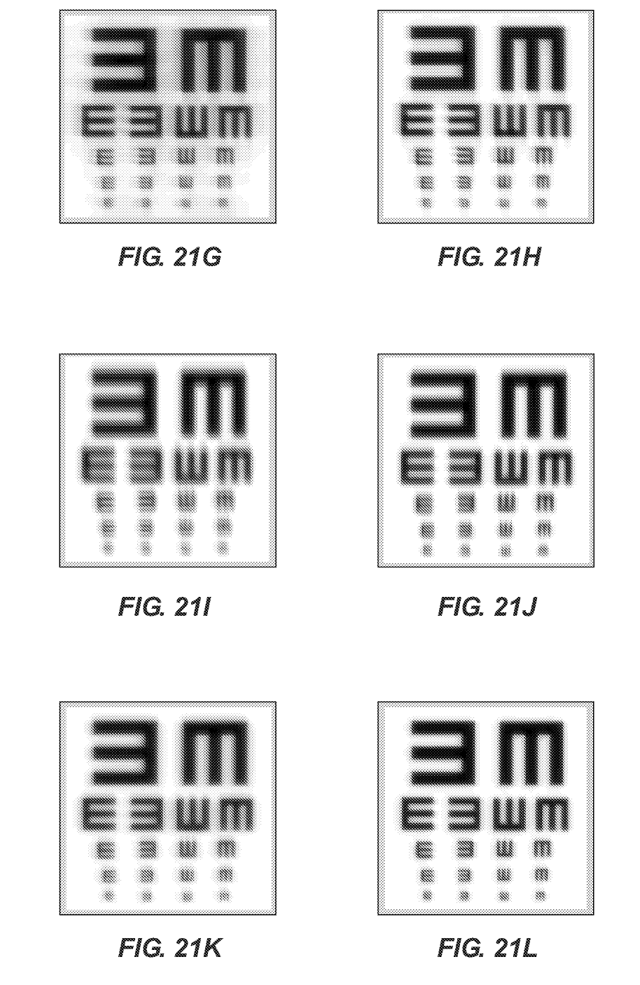

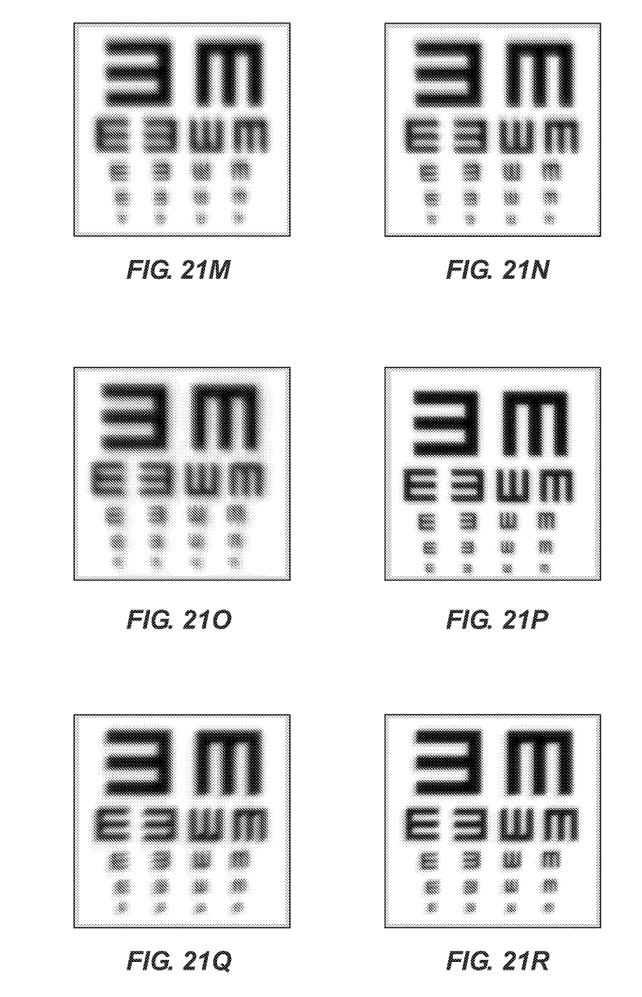

Third, visual acuity improved by one line was found in 9 out of 21 eyes (about 43%). Retinal images on the left (FIG. 21A, 21C, 21E, 21G, 21I, 21K, 21M, 21O, 21Q) are for the 9 eyes with a conventional correction, while retinal images on the right (FIG. 21B, 21D, 21F, 21H, 21J, 21L, 21N, 21P, 21R) are for the corresponding eyes using a lens with wavefront-optimized pupil according to the MIQ optimization.

Fourth, visual acuity improved by more than one line was found in 5 out of 21 eyes (about 24%). Retinal images on the left (FIG. 22A, 22C, 22E, 22G, 22I) are for the eyes with a conventional correction while retinal images on the right (FIG. 22B, 22D, 22F, 22H, 22J) are for the corresponding eyes using a lens with wavefront-optimized pupil according to the MIQ optimization.

Based on the results shown in FIGS. 19A through 22J, we can conclude that visual acuity can be improved in more than 70% of normal eyes when corrected with a lens with wavefront-optimized pupil according to the MIQ optimization. If the contrast factor is taken into account, improvement in visual acuity can be found in far more than 70% eyes because MIQ optimization will give better retinal contrast.

Wavefront-Guided Manifest Refraction and Clinical Refraction for Lenses with Controlled Pupil Transmittance

Vision correction using lenses with controlled pupil transmittance is not practical without a suitable clinical refraction process. A few invention steps can be added to the conventional manifest refraction procedure. First, manifest refraction of human eyes must be performed for a plurality of pupil transmittance profiles or pupil sizes so that an optimized refractive correction can be determined clinically based on the difference of subjective acuity, subjective clarity, and subjective brightness for different corrections. Second, standard phoroptors must be modified for refraction of eyes with controlled pupil transmittance profiles or sizes. Third, a wavefront-guided manifest refraction is desired for a fast comprehensive analysis of vision for symptoms, acuity, clarity, brightness, and for providing an optimized pupil size.

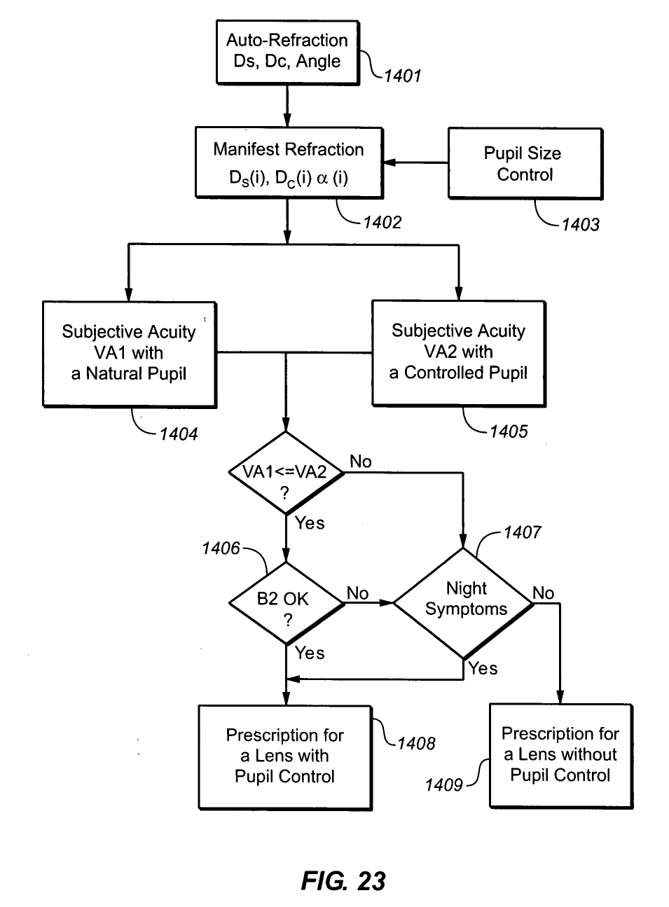

Referring to FIG. 23, a method for prescribing a refractive correction with controlled pupil transmittance through clinical refraction of an eye for a plurality of pupil sizes is shown in block diagram. The method comprises: obtaining an initial sphero-cylindrical correction for an eye 1401 [Ds is spherical power, Dc is cylindrical power, Angle is cylindrical angle], where the initial correction can be from an auto-refractor or from a wavefront aberrometer; determining a manifest refraction of the eye 1402 [Ds(i) is spherical power for different pupil sizes, Dc(i) is cylindrical power for different pupil sizes, α(i) is cylindrical angle for different pupil sizes] and visual acuity of the eye VA1 1404 with an uncontrolled natural pupil and at least another manifest refraction of the eye 1402 and visual acuity VA2 1405 for a reduced pupil size, wherein the reduced pupil size is around 4.3 mm in diameter, or larger than 3.6 mm and smaller than 5 mm; determining an optimized refractive correction based on the measured visual acuities for a plurality of pupil sizes (VA1 and VA2), subjective comparative brightness 1406, and night vision symptoms 1407; providing an optimized prescription as either a refractive correction with pupil control (1408) or as a conventional lens without pupil control.

The brightness 1406 is a subjective judgment of brightness of an acuity chart for a reduced pupil in comparison to that for an uncontrolled pupil. Night symptoms in 1407 can be a subjective assessment of the patient.

The pupil size control 1403 in FIG. 23 is a means to control pupil transmittance in manifest refractions. Controlling pupil size of the subjects in manifest refraction can be achieved in a number of ways including: 1) controlling luminance level of acuity charts, 2) placing contact lenses of different effective apertures on the tested eye, 3) adding a mechanism in the phoroptors to control effective pupil of eye. The method with controlling luminance level is not preferred because the change in luminance level can be as large as 100000 times for a pupil size range between 3.5 mm to 8 mm. The method with contact lens is inconvenient if more than one reduced pupil size is evaluated. Adding a mechanism in the phoroptors to control effective pupil of eye is our preferred embodiment because it has at least two advantages: (1) A number of pupil transmittance (aperture size) can be easily tested; (2) Subject brightness of an acuity chart can be compared under identical luminance conditions.

A method according to the invention for changing aperture size in manifest refraction comprises: a phoroptors with a bank of spherical and cylindrical lenses for refractive corrections; apertures of different sizes placed between the lenses in the phoroptor and the eye for measuring manifest refraction in a plurality of reduced pupil sizes with apertures of various sizes.

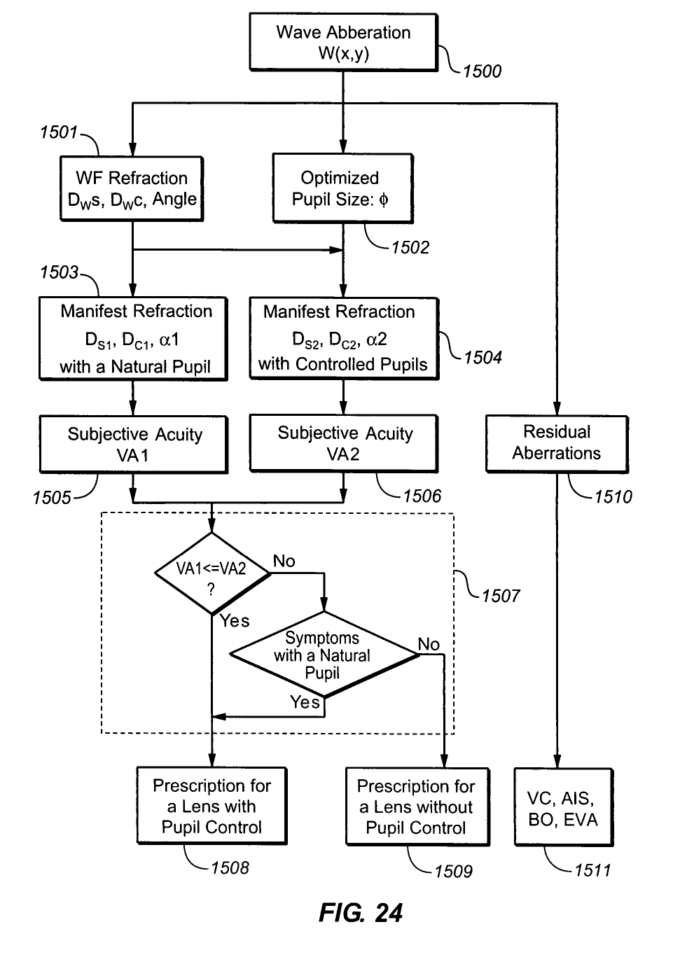

A wavefront-guided manifest refraction in FIG. 24 can solve problems in the conventional manifest refraction. First, a quick wavefront measurement can provide a complete vision analysis of the eye including Vision Clarity (VC), Aberration-Induced Symptoms (AIS), Object Brightness (BO), and even Estimated Visual Acuity (EVA). Second, optical quality for a plurality of pupil size can be obtained easily and compared. Third, a wavefront-optimized pupil size can be determined and used for aiding a quick and accurate selection of an optimized pupil size subjectively. Fourth, vision performance of an eye for a large natural pupil and for a wavefront-optimized pupil can be displayed and compared.

One embodiment for the wavefront-guided manifest refraction provides a comprehensive diagnosis of an eye. According to this embodiment, a method for obtaining the wavefront-guided manifest refraction comprises: obtaining a wave aberration of an eye 1500 from a wavefront measurement using a wavefront aberrometer like a Hartmann-Shack sensor for the eye; determining a wavefront refraction to assist a manifest refraction, wherein the wavefront refraction contains a focus error and a cylindrical error 1501 [Dws is wavefront spherical power Dwc is wavefront cylindrical power, Angle is wavefront cylindrical angle]; determining a manifest refraction of the eye 1503 [Ds1 is manifest spherical power without controlling pupil size, Dc1 is manifest cylindrical power without controlling pupil size, α1 is manifest cylindrical angle without controlling pupil size] and a visual acuity VA1 1505 according to conventional refraction without pupil control; calculating a residual (uncorrected) wave aberration 1510 based on a conventional sphero-cylindrical correction and wave aberration 1500; deriving a complete description of vision performance for the eye 1511 from the uncorrected wave aberration 1510 including Vision Clarity (VC), Aberration-Induced Symptoms (AIS), Object Brightness (BO), and even Estimated Visual Acuity (EVA); providing and displaying a comprehensive refractive diagnosis of the eye with a prescription for a lens 1509 and a complete descriptions of vision based on 1511.

For a lens with a wavefront-optimized pupil, an preferred embodiment for the wavefront-guided manifest refraction, comprising: obtaining a wave aberration of an eye 1500 from a wavefront measurement using a wavefront aberrometer like a Hartmann-Shack sensor for the eye; determining a wavefront refraction to assist manifest refraction, wherein the wavefront refraction contains a focus error and a cylindrical error 1501; determining at least one optimized pupil size to assist manifest refraction with a reduced pupil; determining a manifest refractions of the eye 1503 and visual acuity VA1 1505 for a uncontrolled pupil and a manifest refractions of the eye 1504 [Ds2 is manifest spherical power with controlling pupil size, Dc2 is manifest cylindrical power with controlling pupil size, α2 is manifest cylindrical angle with controlling pupil size] and visual acuity VA2 1506 for a reduced pupil; determining residual (uncorrected) wavefronts for a large uncontrolled pupil and for a reduced pupil 1510 by removing refractive corrections from wave aberration 1500; calculating and comparing vision quality of the eye 1511 for a large uncontrolled pupil and for a reduced pupil, wherein the vision quality of the eye may include Vision Clarity (VC), Aberration-Induced Symptoms (AIS), Object Brightness (BO), and even Estimated Visual Acuity (EVA); selecting a refractive correction between a lens with controlled pupil and a lens without pupil control 1507; providing and displaying a comprehensive refractive diagnosis of the eye with a prescription for a lens 1508 (or 1509) and a complete descriptions of vision based on vision quality in 1511.

The following describes the surprising discovery that photons through pupil periphery of a typical eye at night can generally be characterized as photon noise and method and apparatus to reduce or eliminate photon noise. Extra photons through pupil periphery beyond a pupil diameter of 5 mm for photopic (cone) vision (6 mm for scotopic rod vision) do not increase retinal intensity as commonly expected but reduce retinal image quality for poor visual acuity, reduced vision clarity, and even for causing aberration-induced symptoms.

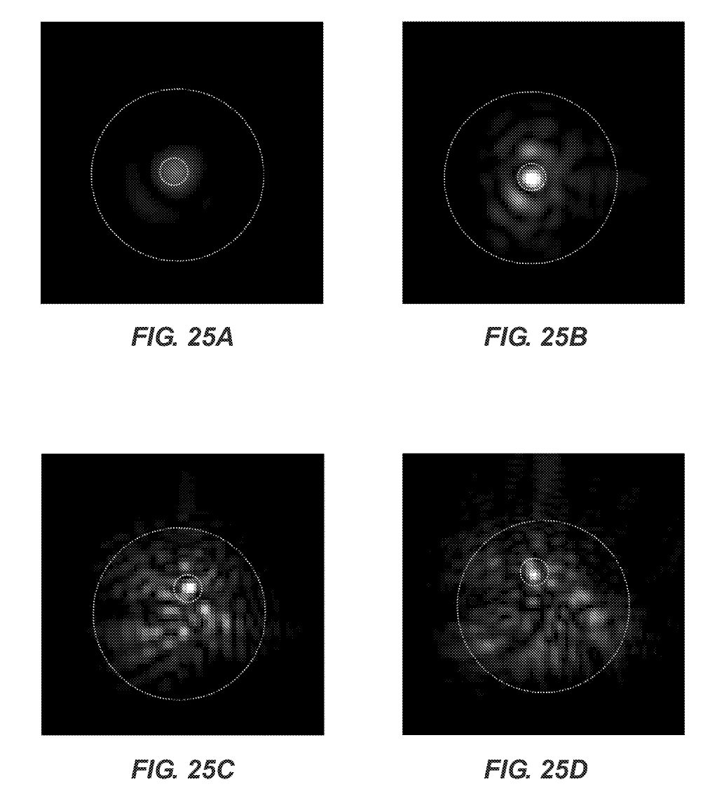

Referring to FIG. 25A-D, retinal point-spread functions of a typical eye for a pupil size of 2 mm (FIG. 25A), 4 mm (FIG. 25B), 6 mm (FIG. 25C), and 7.7 mm (FIG. 25D), respectively, are shown. The total intensity for the point spread function at each pupil size is made proportional to the total photon energy within each pupil size. The point-spread function of the eye is derived from a measured wave aberration of a normal eye with a visual acuity of 20/20. In the calculation, conventional refractive errors such as defocus and astigmatism are removed from the eye's wave aberration because they can be easily corrected with a conventional lens. Standard Stiles-Crawford effect is used as the transmittance function across the pupil of the eye.

Even though the total energy, illustrated by the total intensity within the larger circles in FIG. 25A-D is more with an enlarged pupil at night (FIG. 25C and FIG. 25D), the true intensity of the point object does not increase because high-order aberrations in the eye make those extra photons at pupil periphery spread into a large retinal area. It is not difficult to conclude that more photons with an enlarged pupil at night do not improve detectability of a small point-like object.

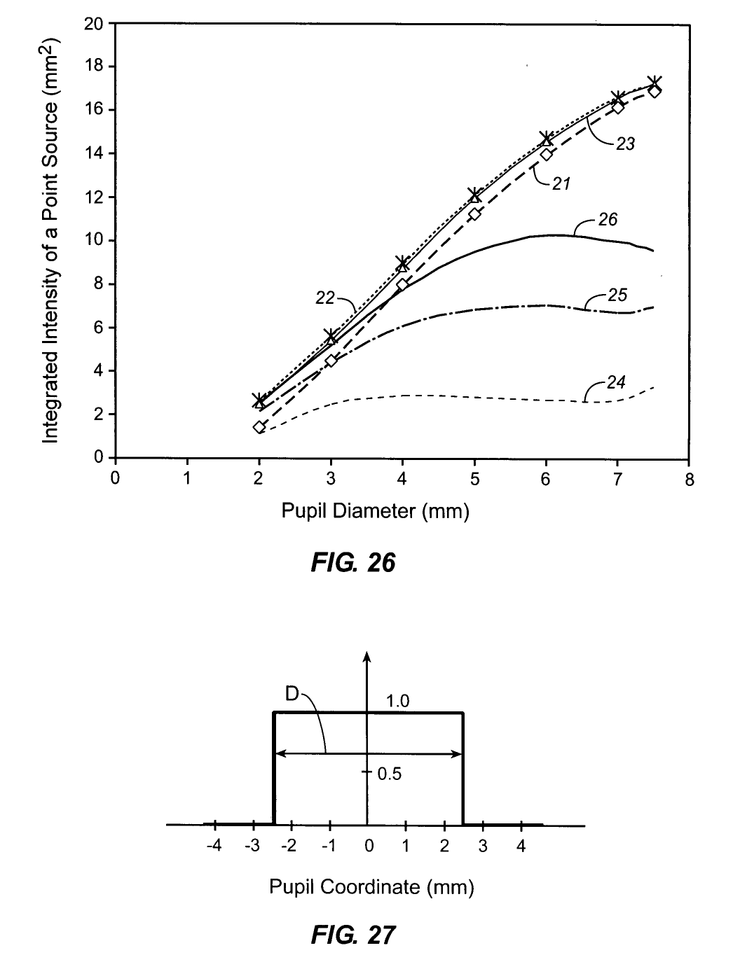

Referring to FIG. 26, a graphical representation of integrated intensity, or total energy over a specified retina area centered at the peak of the point-spread functions (like the small circles in FIGS. 25A-D) for normal human eyes as a function of pupil size is shown. The integrated intensity specifies signal strength of retinal image by taking into account of retinal summation, and also represents the peak intensity of an extended object with uniform intensity. The summation sizes used for the data in FIG. 26 are 1 arc minute (24), 3 arc minutes (25), and 5 arc minutes (26), respectively. As expected, retinal intensity increases as the dimension of retinal summation increases from 1 arcmin (24) to 3 arcmin, and to 5 arcmin (26). Data in FIG. 26 are averaged intensity for 21 normal human eyes with an acuity of 20/20 and better.

As a comparison, we also show the integrated intensity for an ideal aberration-free eye over 1 arc minute (21), 3 arc minutes (22), and 5 arc minutes (23), respectively. For the ideal eye without any aberration, the integrated intensity increases as the pupil size of the eye increases, and does not depend on the size of retinal integration due to the fact that the point spread functions of an aberration-free eye are compact with most energy focused at the image point. However, for the normal human eyes with high-order aberrations, the integrated intensity increases as the pupil size is increased from 2 mm to about 5 mm, but does increase any further beyond a 5 mm pupil. Therefore, photons through pupil periphery of a typical eye beyond pupil diameter of 5 mm do not increase retinal intensity as commonly believed.

With the discovery that increasing pupil size beyond 5 mm does not increase retinal intensity plus the well-known fact that retinal image quality of an eye decreases as pupil size of an eye increases beyond a 4 mm pupil, a variety of improved lenses can be designed to improve vision beyond conventional clear and colored lenses without reducing retinal intensity.

FIG. 27 shows the transmittance profile of a partially opaque lens for improved night vision in accordance to the present invention. The partially opaque lens has at least a transparent inner zone and an opaque outer zone. The transparent inner zone may contain refractive corrections for conventional sphero-cylindrical errors, and the opaque outer zone blocks light energy associating with high-order aberrations in the eye.

In one embodiment, the transparent inner zone has a diameter (D) of around 5 mm to eliminating the noise photons through the pupil periphery for improved night vision.

In another embodiment, the diameter of the transparent inner zone (D) is custom determined according to at least one of the following: to achieve the best retinal image quality in all pupil sizes as described above in connection with FIG. 9., to achieve an optimized retinal image quality and retinal intensity as described above in connection with FIG. 12, and to achieve a therapeutic treatment for a symptomatic eye as described in above in connection with FIGS. 6A-D and FIGS. 7A-D.

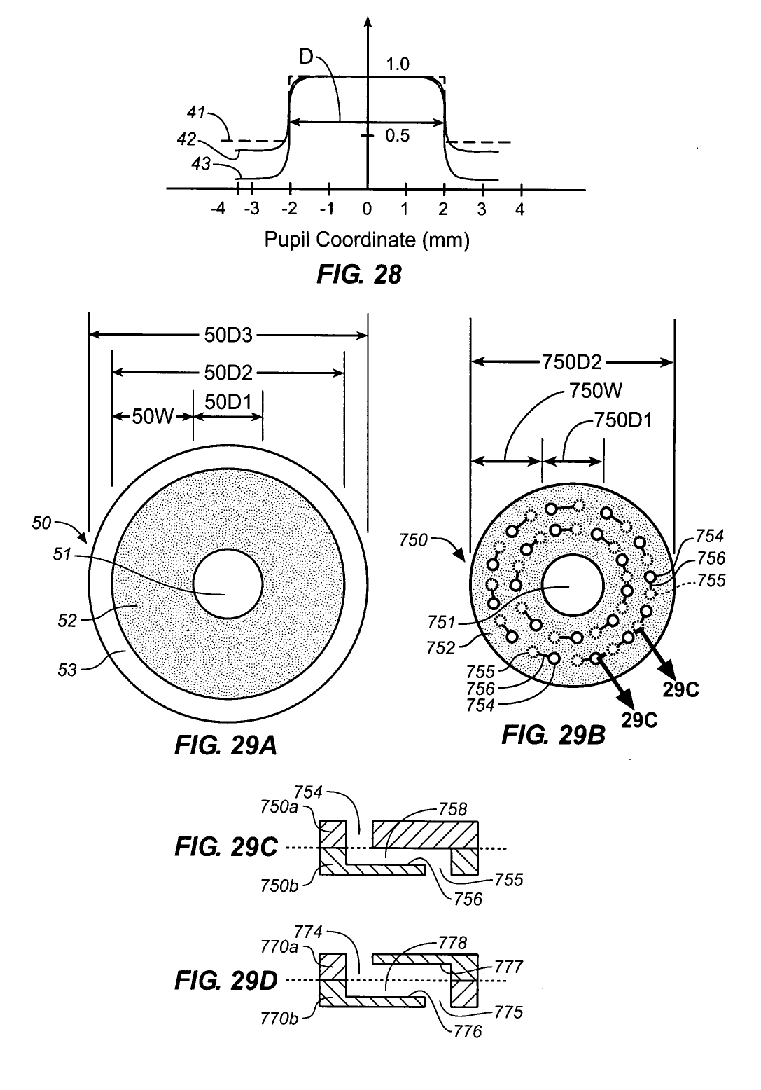

FIG. 28 shows the transmittance profile of a partially attenuated lens for improved night vision in accordance with one embodiment of the invention. The partially attenuated lens comprises at least a transparent inner zone and a partially attenuated outer zone.

Even though the partially attenuated lens does not remove all noise photons like the partially opaque lenses, it will reduce the problems associated with high-order aberrations at night. Additionally, the partially attenuated lens has an advantage that it does not affect the field of view.

For the partially attenuated lenses, the diameter of the inner transparent zone (D) should be determined based on the aberrations in an eye as well as the shape of the transmittance profiles. Example of transmittance profiles include the curves 41, 42, and 43 shown in FIG. 28. And as shown, embodiments illustrated in FIG. 28, have a transmittance greater than 0% and less than 50%. Customization can be made according to at least one of the followings: to achieve a desired retinal image quality, to achieve a balanced retinal image quality as described above in connection with FIG. 12, and to achieve therapeutic treatment for a symptom eye as described in connection with FIGS. 6A-D and FIGS. 7A-D.

Opaque Color Lenses with Improved Night Vision

An opaque color lens 50 to be worn on or implanted in an eye with improved night vision is shown in FIG. 29A, comprising a central non-opaque pupil section 51, an annular colored, opaque iris section 52 to block light entering into the eye beyond the central pupil section, and a clear outside section 53 beyond the annual iris section.

The annular colored, opaque iris section 52 can be a color image of a real eye, or an artistic drawing of a desired iris of an eye.

In one embodiment, the central non-opaque section 51 has a diameter of around 5 mm to eliminating the noise photons through the pupil periphery for improved night vision.

In another embodiment, the diameter of the central non-opaque section 51 is custom determined based on eye's aberrations. Customization can be made according to one of the followings: to achieve the best retinal image quality in all pupil sizes, to achieve a balanced retinal image quality as described above in connection with FIG. 12, to achieve therapeutic treatment as described above in connection with FIGS. 6A-D and FIGS. 7A-D., and to achieve a natural transition between the natural iris of an eye and the color pattern in the annular iris section.

Contact lens 50 according to another embodiment is dimensioned so that 50D1 is 3.25-5.5 mm as supported in Table 1 above (see the minimum and maximum values for MIQ), 50 D2 is 7.0-12.0 mm, 50D3 is about 1 mm more than D2, and 50w is the difference between 50D2 and 50D1 (3.75-8.75 mm). Annular portion 52 comprises material having a light transmittance (1) less than 50% as shown in FIG. 28 or (2) of 5-50% according to another embodiment.

The opaque color lenses with improved night vision can be manufactured with the well-known methods in the art used for making conventional opaque color contact lenses. Well-known methods include those disclosed in U.S. Pat. Nos. 4,582,402, 5,414,477, 6,488,376, and 7,011,408.

In one embodiment according to the invention, the annular colored, opaque iris section is a color image of a desired iris of an eye. Unlike the conventional opaque color contact lenses with a substantial non-opaque portion in the iris section, our opaque color pattern in the iris section is continuous covered with colored or black, opaque material and looks like a natural iris of an eye. In order to ensure that the iris section of the lenses is as opaque as possible, the color pattern may be achieved by two separate steps: one for a color image of a desired iris pattern and the other for a uniform (black or gray) layer to fill-in any non-opaque portion in the first layer.

The opaque color lenses with improved night vision can also be made by sandwiching a color print of a iris pattern between two lens components like the ones disclosed in U.S. Pat. No. 3,536,386, or by using a method like the ones disclosed in U.S. Pat. No. 4,867,552.

Corneal Inlay

Referring to FIG. 29B, a corneal inlay constructed according to principles of the present invention is shown and generally designated with reference numeral 750. Corneal inlay includes an annular portion 752 and a clear portion 751, which can be an opening or a lens configured to provide desired refractive correction. When the clear portion is an opening, the inner circumferential edge of annular portion 752 defines an empty disk shaped space corresponding to clear portion 751. In one embodiment, the inner diameter of annular portion 752 is 3.25 mm-5.5 mm and the outer diameter of annular portion 752 is 6 mm-8 mm. Annular portion 752 comprises material to provide a light transmittance less than 10% or it can be opaque. Annular portion 752 otherwise can have any suitable construction for implantation within a cornea of a human eye including constructions having perforations to allow fluid and/or nutrients such as glucose to pass therethrough as shown in FIGS. 29B-D.

Referring to FIG. 29B, one configuration for fluid and/or nutrient transport from one side to the other side of the inlay is shown. FIG. 29C depicts a sectional view taken along line 29C-29C in FIG. 29B to illustrate one flow channel for a transport hole pair. One hole or recess is formed in one face of the inlay and the other hole or recess of the hole or recess pair is formed in the face that forms the opposite side of the inlay. Each of the other adjacent hole pairs that are aligned in a circumferential direction and are formed in opposite sides of the inlay are coupled through a flow channel of the same construction. More specifically, corneal inlay 750 has two layers or plates 750a and 750b. Layer or plate 750a has a plurality of holes 754 extending therethough and arranged in a multi-circular pattern as shown in FIG. 29B. Each circular array of the multi-circular pattern is at a different radial distance from the center of the inlay. Layer or plate 750b has a plurality of holes 755 extending therethough and arranged in a similar multi-circular pattern. Layer or plate 750b also has a plurality of grooves 756, which are formed in one side or face thereof, extend in a circumferential direction, and are aligned with the multi-circular hole pattern. When layers or plates 750a and 750b are secured to each other, they are arranged so that each hole pair 754 and 755 are circumferentially spaced from one another with channel 756 fluidly coupling the hole pair. In this manner, fluid or nutrients can pass though in the inlay (e.g., the fluid or nutrients can enter hole 754, pass through channel 758 and exit the inlay via hole 775). Since one hole is offset from the other hole in a hole pair and one or both of the layers or plates comprise material that blocks or attenuates light energy, light entering a hole is (1) blocked or (2) attenuated depending on the material selected. As noted above, the material is selected so that the combined layer construction is opaque or provides less than 10% light transmittance.

Referring to FIG. 29D, a variation of the layer or plate configuration of FIG. 29C is shown. Corneal inlay 770 is the same as corneal inlay 750 with the exception that plate 770a also has a plurality of grooves 777 that are aligned with grooves 776 as shown, and grooves 776 are the same as grooves 756. Holes 774 are formed in layer or plate 770a and holes 775 are formed in layer or plate 770b in the same manner as holes 754 and 755 are formed in layer or plates 750a and 750b.

Other known inlay nutrient transport patterns are described in U.S. Patent Application Publication No. 2006/0271176, which is entitled Mask Configured to Maintain Nutrient Transport Without Producing Visible Diffraction Patterns and which published on Nov. 30, 2006. The disclosure of U.S. Patent Application Publication No. 2006/0271176 is hereby incorporated herein by reference.

According to another embodiment of the invention, radial inlay 750 is modified to such that the outer diameter of the annular is about 3.6 mm and the inner diameter of the annular portion is about 1.6 mm. This embodiment provides treatment for presbyopia without night vision correction and minimizes or eliminates light diffusion through the transport holes. The inlay material is selected to provide a light transmittance less than 20% for presbyopia treatments. The nutrient transport patterns would be the same as either of those illustrated in FIG. 29C or 29D or a variation thereof.

Opaque Black Lenses with Improved Night Vision

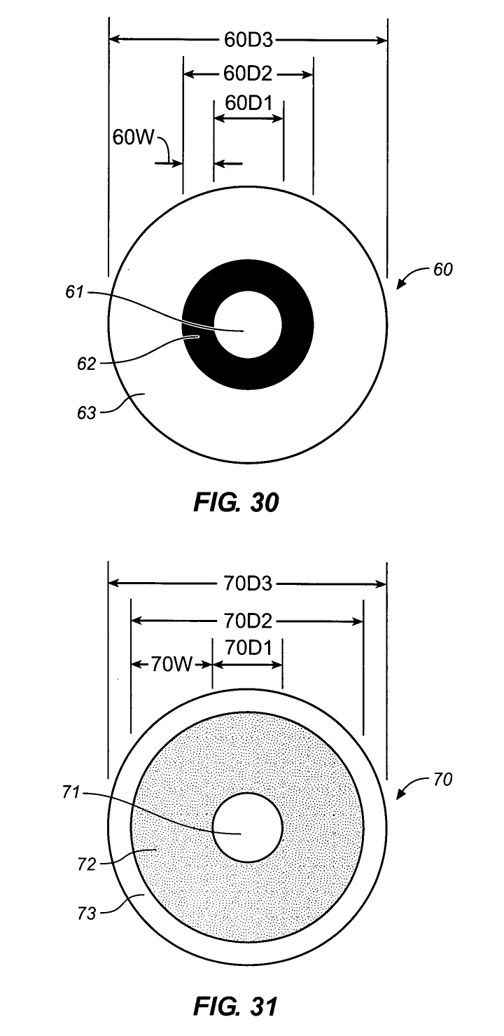

Referring to FIG. 30, another embodiment of an ophthalmic device according to the invention is shown and generally designated with reference numeral 60. Lens 60 can be worn on or implanted in an eye to improve night vision and comprises a central non-opaque pupil section 61, a black, opaque annular iris section 62 to block the light from entering into the eye from beyond the central pupil section, and a clear outside section 63 outside the annual iris section 62.

In one embodiment, the central non-opaque section 61 has a diameter of around 5 mm to eliminating the noise photons through the pupil periphery for improved night vision.

In another embodiment, the diameter of the central non-opaque section 61 is custom determined. Customization can be made according to one of the followings: to achieve the best retinal image quality in all pupil sizes, to achieve a balanced retinal image quality as described above in connection with FIG. 12, and to achieve therapeutic treatment as described above in connection with FIGS. 6A-D and FIGS. 7A-D, and to achieve a natural transition between the natural pupil of an eye and the black annular opaque iris section.

In another embodiment, 60D1 is 3.25 mm to 5.5 mm, 60D2 is 7.0 mm to 8.0 mm, 60D3 is 10 mm to 12 mm, and 60W is 2 mm to 4.75 mm. Transmittance is (1) less than 50% as shown in FIG. 28 or (2) 0% to 50% according to another embodiment.

Typically outer diameter of the black annular opaque iris section is about 8 mm to ensure the periphery of natural pupil at night is total covered by the iris section of the lens. The black opaque lens will not only improve night vision by blocking the photon noise associated with high-order aberrations in the eye but also can enhance the appearance of the eye by enlarging the pupil size in a well-lit situation.

The opaque black lenses with improved night vision can be manufactured using the well-known methods in the art used for making conventional opaque color contact lenses. The well-known method includes those disclosed in U.S. Pat. Nos. 4,582,402, 5,414,477, 6,488,376, and 7,011,408 B2. Instead of a colored pattern for the iris section, the opaque black lens requires to print a uniform black, opaque layer into the lens. Printing multiple black and opaque layers may be used to ensure that the iris section is as opaque as possible in the iris section.

The opaque black lenses with improved night vision can also be made by sandwiching a color print of an iris pattern between two lens components like the ones disclosed in U.S. Pat. No. 3,536,386.

Tinted Color Lenses with Improved Night Vision

Referring to FIG. 31, a tinted color lens adapted to be worn on or implanted in an eye to improve night vision is shown and generally designated with reference numeral 70. Ophthalmic device or lens 70 comprises a central clear pupil section 71, an annular tinted iris section 72, and a clear outside section 73 outside annual iris section 72.

In one embodiment, the central clear section 71 has a diameter of around 4 mm to reduce the noise photons through the pupil periphery for improved night vision.

In another embodiment, the diameter of the central clear section 71 is custom determined based on at least one of the following factors: natural pupil size of an eye, transmittance profile of the annual tinted iris section, and desired retinal image quality at night.

In another embodiment, 70D1 is 3.25 mm to 5.5 mm, 70D2 is 7.0 mm to 8.0 mm, 70D3 is 10 mm to 12 mm, and 70W is 2 mm to 4.75 mm. Transmittance is less than 50% as shown in FIG. 28 or between 5% to 50% in another embodiment.

The annual tinted iris section in one embodiment has a high reflectivity and a low transmittance (less than 50%). High reflectivity in the tinted iris section enables to alter eye's appearance profoundly and low transmittance in the tinted iris section enable to suppress the noise photons through pupil periphery for improved night vision performance.

The tinted color lenses with improved night vision can be manufactured with the well-known methods in the art used for making tinted color contact lenses. These methods include those disclosed in U.S. Pat. No. 4,553,975, U.S. Pat. No. 4,954,132, U.S. Pat. No. 4,891,046, and U.S. Pat. No. 5,939,795, U.S. Pat. No. 5,516,467, and U.S. Pat. No. 6,852,254B2. Adoption of these methods for making tinted color lenses with improved night vision may include some modifications. First, only the iris section is tinted instead of the entire lens. Second, high reflectivity (>50%) in the tinted section can be applied for enhancing iris color more significantly than the conventional tinted lenses.

Transition Contact Lenses with Improved Night Vision

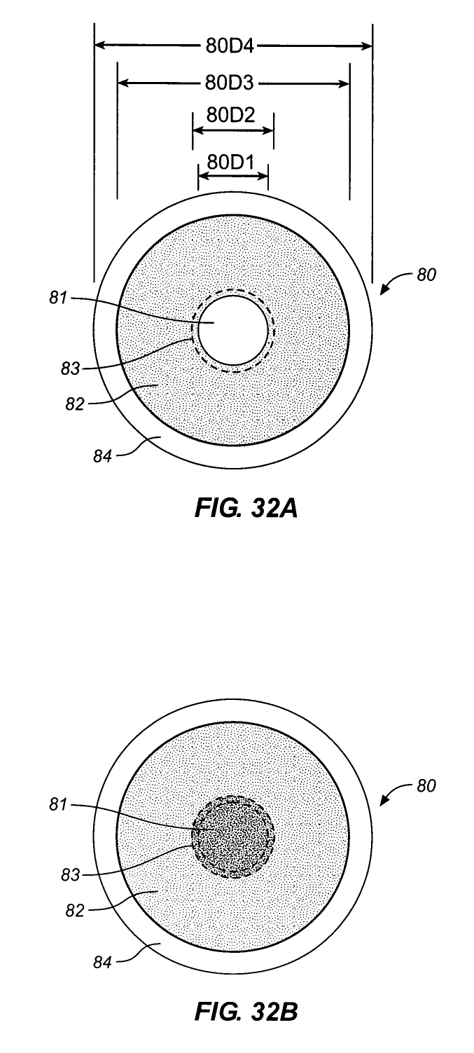

Referring to FIGS. 32A and 32B, a transition contact lens that is configured to improved night vision is shown and generally designated with reference numeral 80. Transition contact lens 80 comprises a central optical section 81, an annular colored, opaque iris section 82 to block light entering into the eye beyond the central optical section, a transition section 83 covering the optical section 81 and part of the opaque iris section 82, and a clear outside section 84 beyond the annual iris section.

FIG. 32A shows the transition contact lens indoors and at night. The transition section 83 is transparent and clear indoors or at night. The entire iris section 82 of the color contact lens is visible indoors and in well-lit situations to viewers for enhanced iris appearance. At night, the opaque section blocks light energy beyond the optical section 81 for improved night vision.

FIG. 32B shows the transition contact lens under sunlight. The transition section 83 automatically darkens to a desired shade and reduces light entering the eye like a sunglass. In a preferred embodiment, the diameter of the transition section 83 is same as or a little larger than that of the central optical section 81. The shaded transition section creates an appearance of an eye with a pupil size as large as the size of the transition section.

The annular colored, opaque iris section 82 can be a color image of a real eye, or an artistic drawing of a desired iris of an eye.

In one embodiment, the central optical section 81 has a diameter of around 5 mm to eliminate the noise photons through the pupil periphery for improved night vision.

In another embodiment, the diameter of the central non-opaque section 81 is custom determined based on eye's aberrations. Customization can be made according to one of the followings: to achieve the best retinal image quality in all pupil sizes, to achieve a balanced retinal image quality, as described above in connection with FIG. 12, to achieve therapeutic treatment as described in connection with FIGS. 6A-D and FIGS. 7A-D, and to achieve a natural transition between the natural iris of an eye and the color pattern in the annular iris section.

In another embodiment, 80D1 is 3.25 mm to 5.5 mm, 80D2 is 7 mm to 8 mm, 80D3 is about 12 mm, 80D4 is about 14 mm and 80W is 6.5 mm to 8.75 mm. Transmittance (1) is less than 50% as shown in FIG. 28 or it (2) is 0% to 50% according to one variation.

The transition contact lenses with improved night vision can be manufactured with the well-known methods in the art used for making conventional photochromic and color lenses. Modifications can be made by a person having ordinary skill in the art.

Method for Prescribing and Producing a Custom Lens with Improved Image Quality

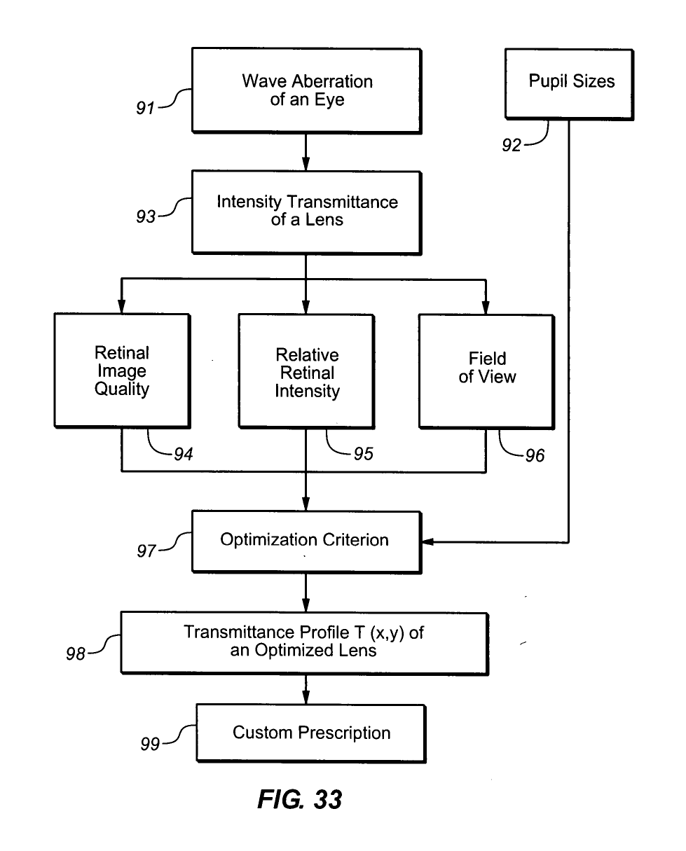

FIG. 33 shows a process for prescribing a custom lens with improved retinal image quality in accordance to the present invention. The method comprises obtaining wave aberration of an eye 91, determining a custom transmittance profile for a custom lens 98, and prescribing a custom lens 99 to achieve a desired vision optimization.

The wave aberration of an eye 91 is measured with a wavefront aberrometer such as a Hartmann-Shack sensor for the eye.

Determining a custom transmittance profile for a custom lens includes calculating at least one of the followings: retinal image quality 94 such as a modulation transfer function, retinal intensity 95 such as an integrated retinal intensity for a point-object, field of view 96; determining an optimized transmittance profile 98 based on a pre-determined optimization criterion 97.

A pre-determined optimization criterion can be one of the followings: to achieve the best retinal image quality in all pupil sizes, to achieve a balanced retinal image quality as described above in connection with FIG. 12., to achieve therapeutic treatment as described above in connection with FIGS. 6A-D and FIGS. 7A-D, and to achieve a natural transition between the natural pupil of an eye and the color pattern in the annular iris section.

Determining a custom transmittance profile for a custom lens may further include vision optimization to eliminate aberration-induced symptoms such as glare, ghost image, and halo.

Determining a custom transmittance profile for a custom lens may further include creating a natural transition for the appearance of the iris of an eye based on natural pupil size of the eye (92).

Prescribing a custom lens 99 to achieve a desired vision optimization include at least specify a transmittance profile of a lens.

Custom lenses can be manufactured with well-known methods in the art according to a custom prescription that specifies at least a transmittance profile.

Intraocular Lenses

The following description relates to intraocular lens embodiments according to the invention with a controlled effective pupil for an eye or controlled light transmittance as described above.

Unlike conventional implanted ophthalmic lenses with an optical zone of 5 mm to 7 mm, intraocular lens embodiments constructed in accordance with principles of the present inventions use an optic lens smaller than 4.5 mm in diameter. This corresponds to about 5 mm at the corneal plane of an eye due to refraction of the corneal surface. As described above, blocking light through pupil periphery beyond a 5 mm pupil does not alter retinal intensity, but can improve night vision significantly and vision of an eye can be further optimized by finding a custom pupil transmittance based on wave aberration of an individual eye. These implantable lenses with a small optic or a small optical section) have many advantages as comparing to conventional intraocular lenses. Among the many advantages are improved night vision where night vision symptoms are reduces or eliminated and/or improved acuity of an eye through custom optimized pupil transmittance by eliminating noise photons at pupil periphery at night. These implantable lenses with a small optics also can provide more consistent vision outcomes because smaller lenses are more tolerable to lens decentration and generally will not be affected by high-order aberrations in an eye. They also make it possible to implant a rigid lens through a small incision, and make accommodation intraocular lenses more practical for small incision procedures, reduced or eliminate the risk of inducing or exacerbating night vision symptoms.

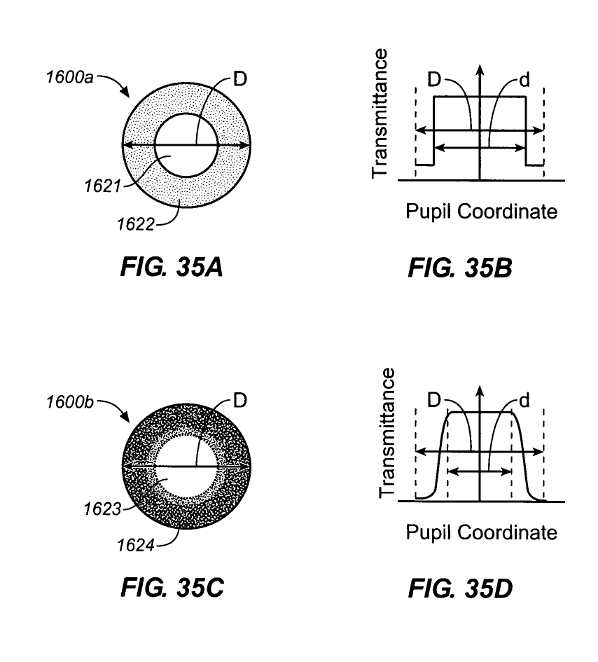

Referring to FIGS. 35A-D, two intraocular lens embodiments are shown.

FIG. 35A shows intraocular lens 1600a, which generally comprises (1) an optic having an inner transparent pupil section 1621 and an outer tinted (or opaque) section 1622 to reduce (or block) photons through the pupil periphery, and (2) haptics (not shown) to fixate the lens inside an eye as is known in the art. The optical lens provides corrections for the refractive errors in the eye, can be a lens of a single focal length or a multifocal lens. FIG. 35B shows a desired transmittance profile for optical lens 1600A. The central clear section inside a diameter of d has a transmittance of 100% while the outer section beyond the central clear section has a transmittance is less than 50%.

In one embodiment, central transparent section 1621 has a diameter (d) of 3.5 mm (or 4 mm pupil at the corneal plane). The outer diameter D of intraocular lens 1600a is between 5 mm and 7 mm (5.7 mm to 7.9 mm at the corneal plane). The intraocular lens in FIG. 35A provides similar vision correction as compared to conventional intraocular lenses during day light when the pupil size of an eye is small, but will reduce or eliminate night vision symptoms caused by high-order aberrations at night when the pupil size of a person's eye is relatively large.

In another embodiment, the diameter of the central transparent section 1621 is custom determined for an individual eye based on at least one of the following: wave aberration of the eye, corneal topography of the eye, or natural pupil size of the eye. Intraocular lenses with such customized effective pupil transmittance can further improve the vision of individual's eyes. Depending on the individual and the customization, they improve acuity and reduce vision symptoms at night.

Intraocular lens 1600a can be manufactured as a rigid lens or a foldable lens using well-known methods in the art. This may involve making the transparent lens first, and tinting the transparent lens to a desired transparency or printing an opaque layer in the desired outer section of a transparent lens.

Referring to 35C another intraocular lens embodiment according to the invention is shown and generally designated with reference numeral 1600b. Intraocular lens 1600b comprises an optical lens with a transparent central section 1623 and an outer section 1624 with gradually decreased transmittance radially to the lens edge, and can include haptics (not shown) to fixate the lens inside an eye as is known in the art.

The transmittance of the lens 1600b is totally transparent in central region 1623, which has a diameter of 3.5 mm, and is reduced gradually towards the edge of the lens to about zero like a half Gaussian profile as shown in the graphic representation of FIG. 35D.

In another embodiment, the transmittance profile of lens 1600b is custom determined for an individual eye based on at least one of the following: wave aberration of the eye, corneal topography of the eye, or natural pupil size of the eye. Intraocular lenses with custom pupil transmittance enable to improve vision of eyes for improved acuity and reduced vision symptoms at night.

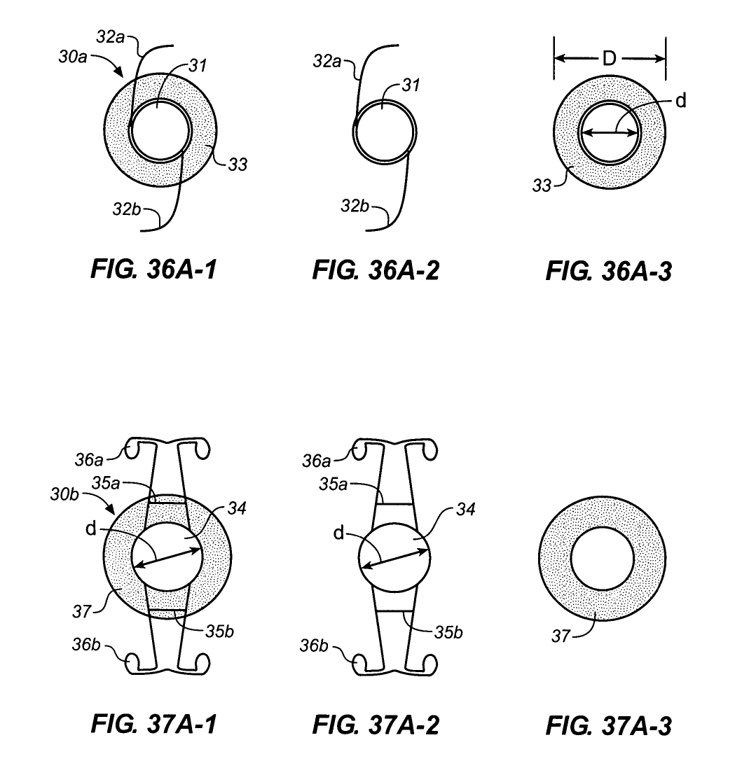

Referring to FIGS. 36A-1, A-2, and A-3, another intraocular lens embodiment is shown and generally designated with reference numeral 30a. Intraocular lens 30a comprises an inner optic section 31 for refractive correction, an outer non-optical opaque section 33 to block light energy from entering through or light energy exiting form (depending on whether it is placed in front of or behind the pupil) the pupil periphery, and haptics 32a,b to fixate the lens inside an eye. The inner optics can be either a lens of a single focal length or a multifocal lens. The haptics can be directly secured to the lens or they can be secured to the lens through an annular extension of the lens as shown in FIG. 36A-2.

In one embodiment, inner optics section 31 is a rigid lens with a diameter of about 3.3 mm and the non-optical section 33 is foldable and firmly attached to the inner optics section. This design has many advantages as compared to conventional rigid lenses. First, it allows a physician to implant a rigid optical lens through a small incision because the outer non-optical section can be made from material that is foldable. Second, the non-optical section blocks photons from entering through pupil periphery or the exit from the pupil periphery (depending on whether it is placed in front of or behind the pupil) to improve night vision of the eye. Third, the designed lens is tolerable to lens decentration and generally or typically will not be affected by high-order aberrations in an eye.

The optic 31 and the haptics 32a and 32b can be made like a conventional rigid lens with flexible haptics using well-known methods in the art. The flexible non-optical section 33 can be made with materials not for conventional lenses but is safe and stable chemically for implants in an eye. For improved safety, the non-optical section can be coated with a thin layer of plastic materials used in conventional intraocular lenses. The non-optical section also can be made from low-grade plastics, tinted to a desired transparency, and coated with high-quality plastics for conventional intraocular lenses. The flexible non-optical section can also be made opaque by sandwiching an opaque layer inside between two flexible layers of material or by tinting a transparent layer. The foldable section can be attached to the rigid lens by fixing them together mechanically or using known methods for making hybrid lenses.

Referring to FIGS. 37A-1, 37A-2, and 37A-3 an accommodation intraocular lens constructed in accordance with the invention is shown and generally designated with reference numeral 30b. Intraocular lens 30b comprises an inner optics section 34 for refractive correction, an outer non-optical section 37 to block light energy through the pupil periphery, haptics sections 36a,b to fixate the lens inside an eye, and hinges 35a,b in the haptics to make the haptic arms flexible. If a rigid lens is used, the focal length of an eye can be adjusted by moving the lens back and forth with the hinges during accommodation control. The eye muscles move the lens back and forth. If a semi-flexible lens is used, focal length of an eye can be adjusted by deforming the lens or by moving the lens back and forth with the hinges.

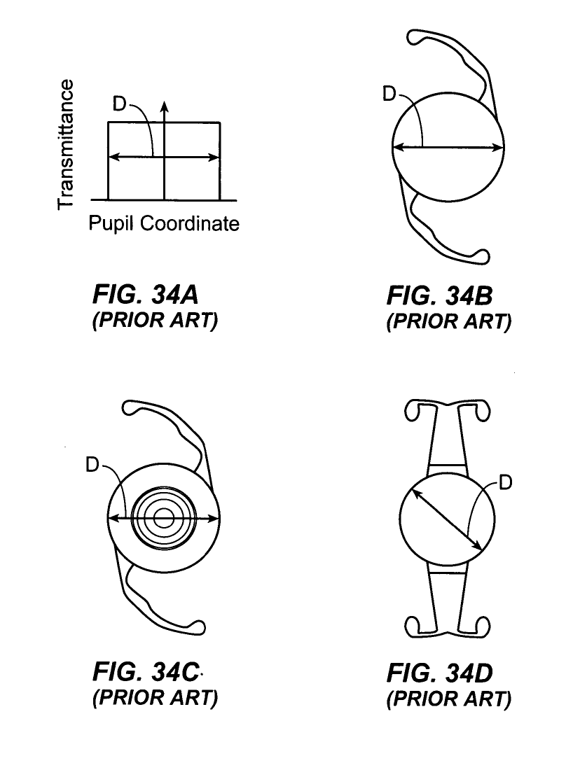

In one embodiment, inner optics section 34 is a rigid or semi-flexible lens with a diameter of around 3.3 mm and the non-optical section 37 is foldable and attached to the optics section. Among the many advantages of this design as compared to the design of FIG. 34D are, (1) it allows the physician to implant a rigid lens or a semi-flexible lens through a small incision when the outer non-optical section is constructed of foldable material, (2) a smaller lens in the optical section can lead to thinner and lighter lens, and (3) the non-optical section can blocks photons from entering through pupil periphery or as they exit from the pupil periphery (depending on the intraocular lens position) to improve night vision of the eye. In a further embodiment the non-optical section allows more diverse designs so that fluid inside the eye can pass through the non-optical section and make lens movement easier. The non-optical section can be made with holes but covered with patches so that fluid can flow through the holes for reduced resistance.

The optics and the haptic section can be made with well-known methods in the art used for making conventional rigid intraocular lenses, but with a smaller optics. The flexible section is non-optical and can be made with materials not for conventional lenses, but is safe and stable chemically for implants in an eye. For improved safety, the non-optical section can be coated with a layer of plastic materials used in conventional intraocular lenses. The non-optical section also can be made low-grade plastics, tinted to a desired transparency, and coated with high-quality plastics for conventional intraocular lenses. The flexible non-optical section can also be made opaque by sandwiching an opaque layer inside the flexible material or by tinting a transparent layer. The foldable section can be attached to the rigid lens by fixing them together mechanically or using the known methods for making hybrid lenses.

Phakic intraocular lens are implanted in front of the iris of an eye, and are partially visible. For Phakic intraocular lenses, the lens appearance often is as important as vision outcomes after implantation. Changes in eye's appearance with a conventional Phakic intraocular lens are often observed clearly at the lens boundary and slightly in iris section behind an intraocular lens even though Phakic intraocular lens is totally transparent.

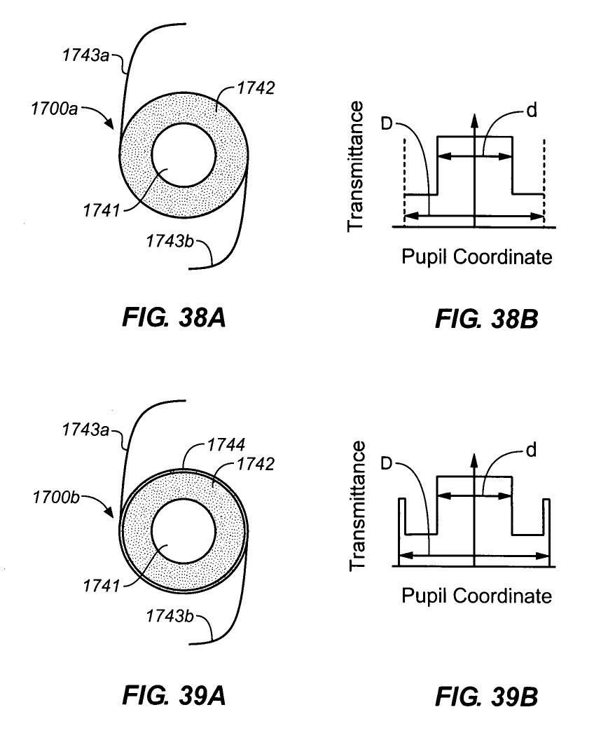

Referring to FIG. 38A, a Phakic intraocular lens in accordance with the invention is shown and generally designated with reference numeral 1700a, comprising a central transparent pupil section 1741, an outside tinted (or opaque) section 1742 to reduce (or block) photons through pupil periphery, and haptics 1743a,b to fixate the IOL onto the iris of an eye. FIG. 38B illustrates a transmittance profile for intraocular lens 1700a.

In one embodiment, central transparent section 1721 has a diameter (d) of about 3.6 mm, corresponding to a 4 mm pupil at the corneal plane. The diameter of the intro-ocular lens (D) is between 5.5 mm and 7.3 mm, corresponding to a 6 to 8 mm pupil at the corneal plane. The transparency of the outside section is preferred to be less than 50% and determined to make this outside section look opaque to viewers so that the Phakic intraocular lens is blended with the natural pupil of an eye for day vision. The appearance of the eye with such a Phakic intraocular lens will not have the same problem of conventional transparent lenses, but will make pupil size of the eye appear as large as the Phakic intraocular lens. At night, the outside section of the Phakic intraocular lens in 38A will reduce or eliminate night vision symptoms caused by high-order aberrations at night when the pupil size of an eye is large.

In another embodiment, the diameter of the central transparent section 1721 is custom determined for an individual eye based on at least one of the following: wave aberration of the eye, corneal topography of the eye, or natural pupil size of the eye. Intro-ocular lenses with customized effective pupil transmittance enable to further improve vision of eyes for improved acuity and reduced vision symptoms at night.

Referring to FIG. 39A, a variation of intraocular lens 1600a is shown and generally designated with reference numeral 1700b. In this embodiment, a second outer section 1744 is added to the intraocular lens and surrounds first outer section 1742. The second outer section 1744 is small in size or thin in the radial direction, and more transparent than the first outer section 1742. Addition of the second outer section 1744 allows to make the transition from the edge of the iris to the Phakic intraocular lens more natural that a sharp transition of color appearance.

Phakic intraocular lenses 1700a and 1700b can be manufactured as a rigid lenses or a foldable lenses using well-known manufacturing methods or techniques. This may involve the steps of making a transparent lens first, and tinting the transparent lens to a desired transparency or printing an opaque layer in the desired outer section of a transparent lens.

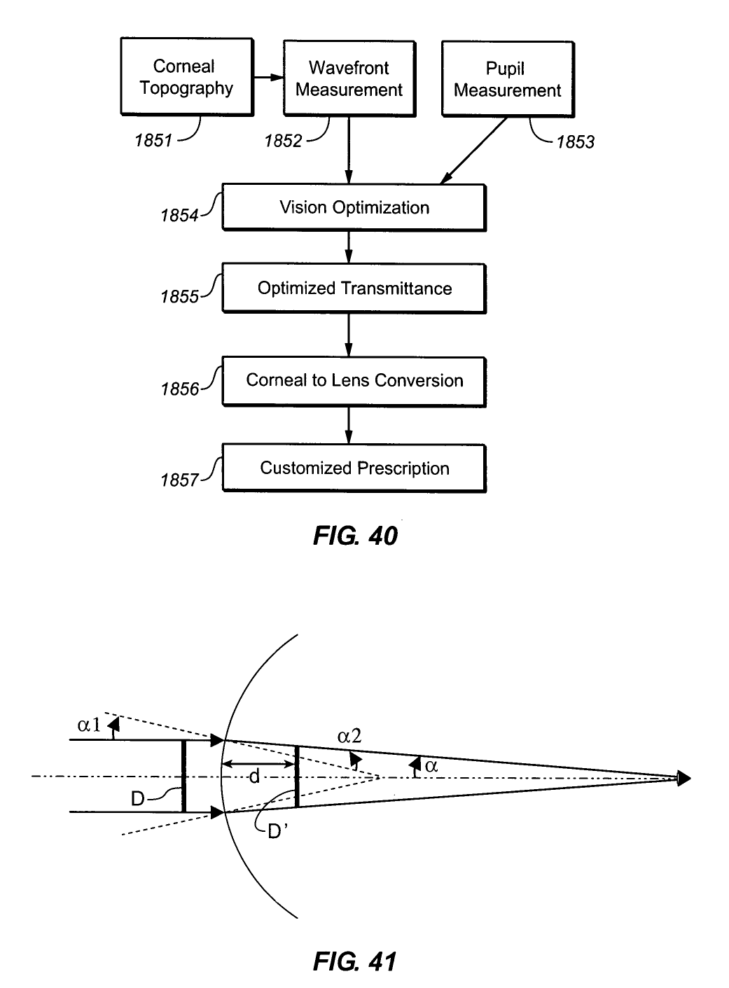

FIG. 40 is a block diagram illustrating a method for optimizing an implantable ophthalmic lens in accordance to the present invention. A transmittance profile of a custom intraocular lens is determined and prescribed based on at least one of the following: corneal topography of an eye 1851, wave aberration of an eye 1852, and pupil size of an eye 1853.

Total wave aberration of an eye (high-order aberrations only) with an intraocular lens determines vision performance of an eye. If the total wave aberration is known and cannot be changed, vision of an eye can be custom optimized by selecting a custom pupil transmittance as described above in connection with FIG. 10 and FIG. 15. An optimized transmittance profile 1855 can be determined from the total wave aberration of an eye through a wavefront optimization 1854 to achieve an optimized retinal intensity and retinal image quality.

If the implanted intraocular lens is for a cataract surgery, the total wave aberration of an eye with the implanted intraocular lens can be calculated with known corneal topography 1851, known in the prior art. Once the total wave aberration of the eye is known from corneal topography, the design of the intraocular lens can be performed the same way as that shown in FIG. 10 and FIG. 15. If the implanted lens is for intraocular contact lenses or a Phakic intraocular lens, the total wave aberration of an eye with an intraocular lens can be approximated with the wave aberration of an eye before refractive correction 51. For wavefront optimization as described above in connection with FIG. 10 and FIG. 15, the aperture size of the intraocular lens is often small and less than 4.5. It is reasonable to assume that introduction of an intra-ocular lens will not add high-order aberrations to the eye, and wave aberration of an eye can be approximated by the wave aberration of an eye measured with a wavefront aberrometer if a Phakic intraocular lens or an intraocular contact lens is concerned. It is also reasonable to assume that high-order aberration of an eye with an intraocular lens for cataract surgery can be approximated by the wave aberration of the cornea. Thus, wave aberration of an eye with an intraocular lens for cataract surgery can be determined from corneal topography and used for wavefront-optimization. Additionally, pupil size of an eye at different light levels can be considered to create a natural appearance of an eye.

If a custom transmittance profile of an eye is determined at the corneal plane in the wavefront optimization, the transmittance profile of an intraocular lens away from the corneal vertex 57 can be obtained by a linear coordinate transformation 56. Suppose the optimized pupil size at the corneal plane is D and the implantable lens is placed d mm away from the corneal vertex. Referring to FIG. 41, the optimized clear central optical section at the lens plane is
D′=D*(L−d)/L

where L is obtained by
L=D/tan(α)

and α is obtained from

α=arcsin(0.5 D/*r)−arcsin(0.5 D/(n*r)),

where r is corneal radius (about 7.8 mm) and n the refraction index of cornea (1.37). For a pupil size of 5 mm at the corneal plane, the size of a Phakic IOL (2.6 mm behind the cornea) is 4.53 mm in diameter and the size of a IOL for cataract (4 m behind the cornea) is 4.28 mm.

Refractive prescription of a custom intraocular lens can be determined from the corneal vertex to the lens aperture using a linear transformation.

In a further embodiment, the inner diameter of the annular portion and the outer diameter of the central clear portion of an intraocular lens can correspond to about 87% of the dimensional values provided in connection with the contact lenses described above (e.g., device 50 of FIG. 29A). Accordingly, the inner diameter of the annular portion for an intraocular lens can be 87% of 3.25 mm, 87% of 5.5 mm or anything between those values, and the outer diameter can be 87% of 8.0 mm.

Variations and modifications of the devices and methods disclosed herein will be readily apparent to persons skilled in the art. As such, it should be understood that the foregoing detailed description and the accompanying illustrations, are made for purposes of clarity and understanding, and are not intended to limit the scope of the invention, which is defined by the claims appended hereto.

That which is claimed is:

1. A method of selecting an ophthalmic device to improve night vision comprising:
obtaining a wave aberration a patient's eye using wavefront analysis; and
selecting a transmittance profile for at least a portion of the device to control light transmittance through the pupil of the eye.

2. The method of claim 1 wherein the ophthalmic device is selected to have a central portion and an annular portion surrounding the central portion where the light transmittance of a region of the annular portion that extends from the central portion is less than the light transmittance of the central portion.

3. The method of claim 2 wherein the annular portion has an inner diameter and the inner diameter of the annular portion is selected to be less than the maximum diameter of the pupil.

4. The method of claim 1 wherein said device is a contact lens.

5. The method of claim 1 wherein said device is an intraocular lens.

6. The method of claim 5 wherein said device is a Phakic intraocular lens.

7. The method of claim 1 wherein said device is a corneal inlay.

8. A method of prescribing an ophthalmic device with controlled optical light transmittance to improve night vision comprising:
obtaining a wave aberration data of a patient's eye;
obtaining a manifest refraction if the eye is myopic;
obtaining a manifest refraction if the eye is hyperopic;
determining the uncorrected aberrations of the eye by removing predetermined aberrations;
selecting a plurality of light transmittance profiles for the device;
calculating optical quality of the eye using complex pupil functions from the determined uncorrected aberrations and the selected light transmittance profiles;
determining a light transmittance profile from the selected light transmittance profiles by optimizing vision between retinal image quality and retinal image intensity;
providing a prescription of an ophthalmic device including a specification of refractive correction if the patient is myopic or hyperopic and the determined light transmittance profile.

9. The method of claim 8 wherein said predetermined aberrations are sphero-cylindrical errors.

10. The method of claim 8, wherein the ophthalmic device is an optical element adapted to be coupled to a patient's eye.

11. The method of claim 10 wherein the optical element is a contact lens.

12. The method of claim 10 wherein the optical element is an a intraocular lens

13. The method of claim 10 wherein the optical element is a Phakic intaocular lens.

14. The method of claim 10 wherein the optical element is a corneal inlay.

15. The method of claim 10, wherein the prescribed specification of pupil light transmittance is described by the sizes of the central clear optical zone and the outer attenuated zone as well as the light transmittance of the outer optical section.

16. A method for determining a light transmittance profile of an ophthalmic device for improving night vision of human eyes comprising:
obtaining a wave aberration of an eye and a manifest refraction if the eye is myopic and hyperopic;
determining the uncorrected aberrations in the eye by removing certain aberrations in the eye;
calculating optical quality of an eye based on the determined uncorrected aberrations;
finding the best corrected optical quality of the eye such as the best MTF in all possible pupil size in a natural pupil and the optical quality of the eye with the larges natural pupil at night without controlling pupil light transmittance;
determining a light transmittance profile for the device that offers an improved night vision quality that ranks between the best corrected optical quality in all possible pupil sizes and the optical quality with the largest natural pupil at night without controlling pupil light transmittance.

17. The method of claim 16, wherein the ophthalmic device is an optical element adapted to be coupled to a patient's eye.

18. The method of claim 17 wherein said ophthalmic device is a contact lens.

19. The method of claim 17 wherein said ophthalmic device is an intraocular lens.

20. The method of claim 17 wherein said ophthalmic device is a Phakic intraocular lens.

21. The method of claim 17 wherein said ophthalmic device is a corneal inlay.

22. A method of prescribing an ophthalmic device with controlled optical light transmittance for improving human vision comprising:
obtaining a manifest refraction of an eye,
measuring optical quality of the eye with a plurality of pupil light transmittances;
determining a light transmittance profile by optimizing vision between retinal image quality and retinal image intensity; and
providing a prescription of an ophthalmic device that contain a specification of light transmittance and refractive correction.

23. The method of claim 22, wherein measuring optical quality of an eye with a plurality of pupil light transmittances comprises of measuring acuity of eye subjectively with at least two pupil light transmittance profiles.

24. The method of claim 22, wherein measuring optical quality of an eye with a plurality of pupil light transmittance comprises of measuring optical quality of an eye objectively using a double-pass point-spread function of an eye.

25. The method of claim 22, wherein measuring optical quality of an eye with a plurality of pupil light transmittance comprises of measuring wave aberration of an eye, calculating optical quality of an eye from the measured wave aberration with a plurality of pupil light transmittance profiles, determining an optimized pupil light transmittance profile based on the calculated retinal image quality.

26. An ophthalmic device for improving night vision comprising a disk shaped member having a clear central optical portion with a diameter that is custom determined based on wave aberrations of a patient's eye and an outer annular portion surrounding the central optical portion and having reduced light transmittance as compared to the clear central optical portion.

27. The ophthalmic device of claim 26 wherein the annular portion has a light transmittance less than 50%.

28. The ophthalmic device of claim 26 wherein the annular portion is sized to cover a portion of the patient's periphery pupil at night.

29. The ophthalmic device of claim 26 wherein the device is a contact lens

30. The ophthalmic device of claim 29 wherein the diameter of the central portion is 3.25-5.5 mm.

31. The ophthalmic device of claim 26 wherein the device is an intraocular lens.

32. The ophthalmic device of claim 31 wherein the central portion has a diameter of 3.0-5 mm.

33. The ophthalmic device of claim 26 wherein the device is a corneal inlay.

34. The ophthalmic device of claim 27 wherein the central portion has a diameter of 3.25 to 5.5 mm

35. The ophthalmic device of claim 26 wherein the central portion is a lens that refracts light.

36. An ophthalmic device for improving night vision comprising a disk shaped member having a clear central optical portion and an annular portion surrounding the central optical portion, said central optical portion having an outer diameter from 3.25-5.5 mm, said annular portion having an outer diameter of 3.75 to 8.75 mm and comprising material that provides light transmittance of 5-50% of visible light to pass therethrough.

37. The ophthalmic device of claim 36 wherein the light transmittance of the annular portion is uniform throughout the annular portion.

38. The ophthalmic device of claim 36 wherein the annular portion is colored.

39. The ophthalmic device of claim 38 further including an outer annular portion surrounding said annular portion, said outer annular portion being clear.

40. The ophthalmic device of claim 36 further including an outer annular portion surrounding said annular portion, said outer annular portion being clear.

41. The ophthalmic device of claim 36 wherein the device is a contact lens.

42. An intraocular lens comprising an optic portion and at least one haptic, said optic portion consisting of a central clear optical section adapted to focus light toward a retina of an eye and a an annular section comprising material having properties such that the annular section transmittance is 5-50%.

43. The intraocular lens of claim 42 wherein all of the entire annular section transmittance is 5-50%.

44. An intraocular lens comprising:
at least one haptic; and
an optic comprising a central optical clear section and an annular opaque section, said central optical clear section having an outer diameter of 3.3 mm to 4.5 mm and being adapted to focus light toward a retina of an eye, said annular opaque section surrounding said clear section to block photons of visible light from passing therethrough the central clear optical section.

45. The intraocular lens of claim 44 wherein said intraocular lens is an accommodating intraocular lens to allow a change the focus power toward the retina of the eye.

46. The intraocular lens of claim 44, wherein the outer opaque section comprises a thin film opaque coating.

47. The intraocular lens of claim 44, wherein the outer opaque section is fluid permeable.

48. An ophthalmic device comprising a member configured and sized to be implanted between the anterior corneal surface and the iris of a patient's eye to improve night vision, said member having an annular configuration with an inner diameter of 3.6 mm to 5 mm and comprising material that attenuates light energy.

49. The ophthalmic device of claim 48 further including a clear lens, said annular member surrounding said lens.

50. The ophthalmic device of claim 49 wherein the clear lens has refractive power to provide correction of refractive errors in the patient's eye.

51. The ophthalmic device of claim 48 wherein said annular member comprises a material having a light transmittance less than 10%.

52. The ophthalmic device of claim 51 wherein the material light transmittance is uniform throughout the annular member.

53. The ophthalmic device of claim 51 wherein the material light transmittance gradually reduces in a radially inward direction.

54. The ophthalmic device of claim 48 wherein the inner diameter of the annular member is custom determined based on wave aberrations in the patient's eye.

55. The ophthalmic device of claim 48 wherein the device is a corneal inlay that is sized and configured for implantation within the cornea of a human eye.

56. A corneal inlay comprising and annular member configured and sized for implantation in a cornea of a human patient, said annular member comprising two layers of material and having an anterior face and a posterior face and a plurality of hole pairs, each hole pair having a first hole partially extending into said annular member from said anterior face and a second hole partially extending into said annular member from said posterior face and one hole of each hole pair being formed in one layer of said two layers and the other hole of each hole pair being formed in the other layer of said two layers, said annular member having a plurality of channels formed therein, each channel fluidly coupling the holes of a hole pair.

57. The corneal inlay of claim 56 wherein each hole of a hole pair has a center axis, and the center axes of a hole pair are not coincident.

58. The corneal inlay of claim 56 wherein the holes of each hole pair are circumferentially spaced from one another.

59. The corneal inlay of claim 56 wherein said annular member has in inner diameter, which is 3.25-5.0 mm.

60. The corneal inlay of claim 56 wherein said annular member has an inner diameter and an outer diameter, the inner diameter is about 1.6 mm and the outer diameter is about 3.6 mm.
